(19) United States
(12) Patent Application Publication
(10) Pub. No.: US 2011/0039950 A1
(43) Pub. Date: Feb. 17, 2011

(54) Alkyl and/or Alkenyl Ethers of Alkyl and/or Alkenyl (Poly)Glycosides and Their Use

(75) Inventors: Ansgar Behler, Bottrop (DE); Achim Ansmann, Erkrath (DE); Catherine Weichold, Aachen (DE); Frank Clasen, Hilden (DE); Anja Wick, Hilden (DE); Eike Ulf Mahnke, Velbert (DE); Bjoern Klotz, Erkrath (DE); Carsten Neumann, Duesseldorf (DE); Matthias Hloucha, Koln (DE); Rolf Kawa, Monheim (DE); Petra Schulte, Koln (DE)

Correspondence Address: FOX ROTHSCHILD LLP, 997 Lenox Drive, Bldg. #3, Lawrenceville, NJ, 08648, US

(73) Assignee: COGNIS IP MANAGEMENT GMBH, Duesseldorf (DE)

(21) Appl. No.: 12/937,821

(22) PCT Filed: Apr. 6, 2009
(86) PCT No.: PCT/EP2009/002511

§ 371 Date: Oct. 14, 2010

(30) Foreign Application Priority Data
Apr. 14, 2008 (EP) 0800729.1
Nov. 7, 2008 (EP) 08019490.5

(51) Int. Cl. A61K 47/26; A61K 8/92

(52) U.S. Cl. 514/777

(57) ABSTRACT

The present invention relates to nanoemulsions comprising alkyl and/or alkenyl ethers of alkyl and/or alkenyl (poly)glycosides, to a method for their preparation and also to their use. The present invention further relates to the alkyl and/or alkenyl ethers of alkyl and/or alkenyl (poly)glycosides themselves, and their use.

### FIELD OF THE INVENTION

The invention is in the field of cosmetic and/or pharmaceutical preparations and relates to alkyl and/or alkenyl ethers of alkyl and/or alkenyl (poly)glycosides, mixtures thereof, and their use, in particular in nanoemulsions. The invention further relates to novel nanoemulsions and to their use. The invention further relates to novel solubility promoters (solubilizers) with an increased dissolving capacity, in particular for fat-soluble active ingredients and UV photoprotective filters.

### STATE OF THE ART

Nanoemulsions are usually understood as meaning emulsions whose particle size or droplet size is below 1000 nm. In particular, nanoemulsions are understood as meaning those with an average particle size of from about 5 to 500 nm. On account of their advantageous properties, nanoemulsions are often used in cosmetic and pharmaceutical preparations. The phase stability even at low viscosities and also the significantly increased resorption rate for active ingredients which are applied with the emulsion for example to skin or hair compared with conventional emulsions are especially advantageous.

Stable nanoemulsions have hitherto been obtained almost exclusively in accordance with the phase inversion method (PIT method). However, only ethoxylated emulsifiers can be used in this method. However, these are often irritative to skin and consequently disadvantageous.

The object of the present invention was to provide nanoemulsions which can be stably prepared as far as possible without ethoxylated emulsifiers. Moreover, it was of interest to provide sensorally improved nanoemulsions. Furthermore, the preparation of the nanoemulsions should be simple and favorable in terms of energy. In particular, it was of interest to provide nanoemulsions which can be prepared by the phase inversion method (PIT method) although they do not comprise ethoxylated emulsifiers. It was a further object to provide nanoemulsions which have a high fraction of internal phase, in the case of O/W emulsions thus a high fraction of oil bodies. Furthermore, it was desirable to provide nanoemulsions with the lowest possible fraction of emulsifiers. Furthermore, it should be possible to prepare the nanoemulsions with a broad spectrum of oil bodies.

It has been found that nanoemulsions according to claim 1 achieve these objects.

Lipophilic substances, such as, for example, vitamins, perfume oils or UV photoprotective filters, can often only be incorporated with difficulty into cosmetic or pharmaceutical preparations, especially if these preparations have a predominantly polar character. In such cases, solubility promoters are used, which are individual substances or mixtures with average HLB values which thus to a certain extent form a bridge from the polar environment to the nonpolar substrate. The terms solubility promoter, dissolution promoter and solubilizer are used synonymously. Very effective solubility promoters are the sulfonates of short-chain alkylaromatics, such as, for example, toluenesulfonate or cumenesulfonate, although, on account of their inadequate skin cosmetic compatibility, they are of no importance in the field of cosmetics and pharmacy. Other cosmetic solubilizers, such as, for example, special hydrophilized oils, are skin compatible, but have inadequate dissolving capacity and/or poor low-temperature behavior, i.e. exhibit the tendency to cloud even at room temperature. These hydrophilized oils are commercially available, for example, under the INCI name “Ethoxylated Hydrogenated Castor Oil”. For this reason, especially in the cosmetics industry, there is a desire for novel solubility promoters (solubilizers) which are free from the disadvantages described above.

A further object of the present invention was therefore to provide novel solubility promoters (solubilizers) which, compared to products of the prior art, have an improved dissolving capacity, in particular toward lipophilic substances, such as, for example, perfume oils, vitamins, UV photoprotective filters, lipophilic pharmaceutical active ingredients and the like. It was of particular interest here that the products are liquid at room temperature. It was furthermore desirable that the products comprise no polyglycol ethers.

### SUBJECT MATTER OF THE INVENTION

The invention provides nanoemulsions comprising a water phase and an oil phase, characterized in that they comprise at least one alkyl and/or alkenyl ether of alkyl and/or alkenyl (poly)glycosides of the formula (I-A)

(Gₘ-R¹)R²ₙ (I-A)
in which G is a sugar radical having 5 or 6 carbon atoms,
R¹ is a C6 to C22 alkyl and/or alkenyl radical in acetal bond,
R² is a C1 to C4 alkyl and/or alkenyl group in ether bond,
m is an average value from 1.0 to 3.0, preferably 1.2 to 1.8, and
n is a number from 0.5 to 5.0, preferably 1.4 to 2.6.

The invention further provides alkyl and/or alkenyl ether mixtures of alkyl and/or alkenyl polyglycosides of the formula (I-C)

(Gₘ-R¹)R²ₙ (I-C)
in which G is a sugar radical having 5 or 6 carbon atoms,
R¹ is a C6 to C22 alkyl and/or alkenyl radical in acetal bond,
R² is a C1 to C4 alkyl and/or alkenyl group in ether bond,
m is an average value from 1.2 to 1.8, and
n is a number from 1.4 to 2.6,
where at least 50% by weight of the alkyl and/or alkenyl ethers comprise a radical R¹ with a carbon chain greater than or equal to 12.

Alkyl ether mixtures of alkyl and/or alkenyl polyglycosides are described in the prior art. However, compounds according to formula (I-C) are not disclosed, nor is their suitability as solubilizers.

EP 0 364 852 A2 (BASF) describes substituted glucosides of the formula (Gluₘ-R¹)Rₙ², where Glu is a glucose unit, R¹ is a C8 to C18 alkyl radical in acetal bond, R² is C1 to C4 alkyl groups with ether bond. m has an average value of from 1 to 10 and n has an average value of from 0.1 m to 2 m. The compounds disclosed in EP 0 365 285 A2 carry at most 2 mol of alkyl groups per glucose unit since n is at most 2 m. The starting materials given are glucosides with a value m of from 1 to 10, preferably from 1 to 5, in particular 1.5 to 3 (EP 0 364 852 A2, p. 2, l. 36 and l. 53). The examples in EP 0 364 852 A2 all have an average value of m of from 2.6 to 2.8. The products described in EP 0 364 852 A2 carry, as radical R¹, alkyl groups with a carbon chain length of from 8 to 18 carbon atoms, the examples according to the invention are glucosides of C10/C12 alkanol distillation steps. In contrast to this, at least 50% by weight of the alkyl ethers of alkyl and/or alkenyl polyglucosides according to the invention have R¹ radicals, which are an alkyl radical having greater than or equal to 12 carbon atoms. Surprisingly, it has been found that by selecting alkyl ethers of alkyl and/or alkenyl polyglucosides with this carbon chain length, improved solubilizers compared with the prior art are obtained.

US 2004/0254084 (McCall) describes in claim 1 a compound made of 2 glucose units which carries an alkyl group having 11 carbon atoms on the C-1 atom. Of the 7 OH groups freely available in this diglucoside, according to the formula in claim 1, 4 OH groups have been etherified with alkyl groups of chain length C1 to C8. Accordingly, the compounds described here have a theoretical degree of polymerization m of 2 and carry exclusively radicals R1 with a carbon chain length of 11.

WO 93/06115 (Henkel) describes an anhydrous method for the preparation of alkyl and/or alkenyl polyglycoside ethers in which alkyl and/or alkenyl polyglycosides of the formula R¹—O—[G]ₚ, in which R¹ is an aliphatic, linear or branched hydrocarbon radical having 6 to 22 carbon atoms and 0, 1, 2 or 3 double bonds, and [G] is a sugar radical having 5 or 6 carbon atoms and p is numbers from 1 to 10, are reacted with halogenated hydrocarbons in the presence of alkaline compounds. The halogenated hydrocarbons described are alk(en)yl halides having 1 to 18 carbon atoms and also benzyl halides (p. 4). According to WO 93/06115 (p. 5, 2^{nd} paragraph), alkyl and/or alkenyl glycosides and halogenated hydrocarbons are used in the molar ratio from 1:0.9 to 1:10, optimally in the molar ratio from 1:1 to 1:5. Although the alkylating agents described in WO 93/06115 on p. 4, 3^{rd} paragraph are also alkyl halides, such as, for example, methyl chloride, the disclosure in WO 93/06115 does not make it possible to obtain compounds according to the invention as in formula (I): the examples describe exclusively benzyl ethers which have been obtained by reacting C12/C14 cocoalkylglucoside with benzyl chloride. Benzyl chloride is present in liquid form at room temperature; in contrast to this, the alkylating agents methyl chloride or ethyl chloride are gaseous at room temperature. If then, as described in WO 93/06115, anhydrous conditions are used, the starting material (alkyl and/or alkenyl polyglycoside), which is likewise present in solid form at room temperature, must be heated to ca. 80° C., and then alkaline conditions established by adding NaOH. This gives a high viscosity mixture from which it is not possible to obtain products according to the invention together with the gaseous alkylating agent. I.e. although the description in WO 93/06115 also describes a reaction with alkylating agents as a process variant, the person skilled in the art can derive from this prior art reworkable teaching only for the aralkylation reaction and thus also only for the aralkylated alkyl and/or alkenyl polyglycosides.

U.S. Pat. No. 4,663,444 (Egan) describes ethers of sugars (monoglucoside) which are substituted at O1 and O6 position, where the alkyl group at O6 position carries 12 to 18 carbon atoms.

### Nanoemulsion

According to the invention, the term nanoemulsion is to be understood as meaning emulsions with a particle size or droplet size of less than 1000 nm. Usually, the particle size of the nanoemulsion according to the invention is in the range from 5 to 500 nm and in particular 50 to 200 nm, preferably 10 to 100 nm.

Alkyl Ethers of Alkyl and/or Alkenyl Polyglycosides and Mixtures Thereof

The nanoemulsion according to the invention comprises at least one alkyl and/or alkenyl ether of alkyl and/or alkenyl (poly)glycosides of the formula (I-A)

(Gₘ-R¹)R²ₙ (I-A)
in which G is a sugar radical having 5 or 6 carbon atoms,
R¹ is a C6 to C22 alkyl and/or alkenyl radical in acetal bond,
R² is a C1 to C4 alkyl and/or alkenyl group in ether bond,
m is an average value from 1.0 to 3.0, preferably 1.2 to 1.8, and
n is a number from 0.5 to 5.0, preferably 1.4 to 2.6.

In a preferred embodiment of the invention, the nanoemulsion comprises at least one alkyl and/or alkenyl ether mixture of alkyl and/or alkenyl (poly)glycosides of the formula (I-B)

(Gₘ-R¹)R²ₙ (I-B)
in which G is a sugar radical having 5 or 6 carbon atoms,
R¹ is a C6 to C22 alkyl and/or alkenyl radical in acetal bond,
R² is a C1 to C4 alkyl and/or alkenyl group in ether bond,
m is an average value from 1.0 to 3.0, preferably 1.2 to 1.8, and
n is a number from 0.5 to 5.0, preferably 1.4 to 2.6,
   where at least 50% by weight of the alkyl and/or alkenyl ethers comprise a radical R¹ with a carbon chain greater than or equal to 12.

A preferred embodiment of the invention relates to a nanoemulsion comprising a water phase and an oil phase, characterized in that it comprises at least one alkyl and/or alkenyl ether mixture of alkyl and/or alkenyl polyglycosides of the formula (I-C)

(Gₘ-R¹)R²ₙ (I-C)
in which G is a sugar radical having 5 or 6 carbon atoms,
R¹ is a C6 to C22 alkyl and/or alkenyl radical in acetal bond,
R² is a C1 to C4 alkyl and/or alkenyl group in ether bond,
m is an average value from 1.2 to 1.8, and
n is a number from 1.4 to 2.6,
where at least 50% by weight of the alkyl and/or alkenyl ethers comprise a radical R¹ with a carbon chain greater than or equal to 12.

G is a sugar radical (=monosaccharide) having 5 or 6 carbon atoms. Of suitability are aldoses, alduloses, hexoses and hexyloses. By way of example, aldoses having five carbon atoms (=pentoses) which may be mentioned are xylose, lyxose, ribose and arabinose. By way of example, aldoses having 6 carbon atoms (=hexoses) which may be mentioned are glucose, galactose or mannose. By way of example, ketoses with an unbranched chain of 6 carbon atoms (=hexyloses) which may be mentioned are fructose or sorbose. Among the aldoses, particular preference is given to glucose. Accordingly, a preferred embodiment of the invention relates to alkyl ethers and alkyl ether mixtures of alkyl and/or alkenyl (poly)glucosides.

The compounds according to the invention are mixtures of alkyl and/or alkenyl ethers of alkyl and/or alkenyl polyglycosides. The alkyl ethers present in the mixture differ by virtue of the degree of polymerization of the sugar units. The number m in the general formula (I) gives the degree of polymerization, i.e. the distribution of monoglycosides and oligoglycosides. Whereas m in a given individual compound must always be an integer, the value m for a specific alkyl and/or alkenyl polyglycoside (which constitutes a mixture of different monoglycosides and oligoglycosides) is an analytically determined parameter which is in most cases a fraction. The degree of polymerization m of the alkyl ethers of alkyl and/or alkenyl polyglycosides according to the invention is 1.0 to 3.0, preferably 1.1 to 2.5, in particular 1.1 to 2.0, preferably 1.2 to 1.8, preferably 1.2 to 1.5, in particular 1.2 to 1.4. If the degree of polymerization m=1, this is a pure monosugar and thus an alkyl ether of an alkyl and/or alkenyl monoglycoside. This is illustrated by the name alkyl ethers of alkyl and/or alkenyl (poly)glycosides, which includes both the monoglycoside and also the polyglycosides.

The degree of polymerization m of the alkyl and/or alkenyl (poly)glycosides to be used as starting materials and also of the alkyl and/or alkenyl ethers of the alkyl and/or alkenyl (poly)glycosides according to the invention can be determined analytically, as described, for example, in “Alkyl Polyglycosides”; K. Hill, W. v. Rybinski, G. Stoll (Ed.), VCH Weinheim, 1997, pp. 23-38.

The number n determines the degree of alkylation of the alkyl and/or alkenyl ethers of alkyl and/or alkenyl (poly)glycosides. This is defined as the ratio of moles of alkoxy groups in the mixture to moles of alkyl and/or alkenyl ethers of the alkyl and/or alkenyl (poly)glycosides. The degree of alkylation of the compounds according to the invention can be determined by quantifying the alkoxy groups and placing them relative to the alkyl and/or alkenyl ethers of the alkyl and/or alkenyl (poly)glycosides. The determination of the alkoxy groups can be carried out, for example, in accordance with the method from Hodges described in: “Quantitative Organic Microanalysis”, Al Steyermark, 2^{nd} Ed., 1961, Academic Press New York/London; pp. 422-424, or in accordance with the DIN method DIN EN 13268. The degree of alkylation of the alkyl and/or alkenyl ethers of alkyl and/or alkenyl (poly)glycosides according to the invention is 0.5 to 5.0, preferably 0.8 to 4.0, in particular 1.0 to 3.0, preferably 1.4 to 2.6, in particular 1.5 to 2.5, preferably 1.5 to 2.0, in particular 1.5 to 1.9.

A further characteristic for describing the degree of alkylation is the OH number (hydroxy number), which indicates how many milligrams of potassium hydroxide are equivalent to the amount of acetic acid which is bonded by 1 g of substance during the acetylation and is thus a measure of the free OH groups. The OH number can be determined in accordance with the DFG method DGF C-V 17a.

The radical R¹ is a C6 to C22, preferably C8 to C18, alkyl and/or alkenyl radical in acetal bond, where R¹ may optionally be hydroxy-substituted, where R¹ may be linear or branched. The radical R² is a C1 to C4 alkyl and/or alkenyl group in ether bond which may optionally be branched. If the radical R² is a C1 to C4 alkyl group, alkyl ethers of alkyl and/or alkenyl (poly)glycosides are obtained. The radical R² can likewise be a C1 to C4 radical which is unsaturated. Alkenyl ethers of alkyl and/or alkenyl (poly)glycosides are then obtained. Particular preference is given to compounds of formula (I) in which R² is a C1 to C4 alkyl radical.

In a preferred embodiment of the invention, the nanoemulsion comprises alkyl and/or alkenyl ether mixtures of the general formula (I-B) or (I-C) in which at least 50% by weight of the alkyl and/or alkenyl ethers comprise a radical R¹ with a carbon chain greater than or equal to 12. In a preferred embodiment of the invention, more than 50% by weight of the ethers (=alkyl and/or alkenyl ethers of alkyl and/or alkenyl (poly)glycosides) carry a radical R¹ with a carbon chain greater than or equal to 12, preferably more than 60% by weight, in particular more than 70% by weight, preferably more than 75% by weight. The % by weight refer to the total amount of the alkyl and/or alkenyl ether mixture.

In one embodiment of the invention, the ether mixtures according to the invention comprise ethers where R¹=C12 (=C12 radical) and R¹=C14 (=C14 radical), and the sum of the alkyl and/or alkenyl ethers where R¹=C12 and C14 constitutes more than 50% by weight, in particular more than 60% by weight, preferably more than 70% by weight, in particular more than 75% by weight, preferably more than 80% by weight, based on the total amount of the alkyl and/or alkenyl ether mixture.

The term “C12 radical” comprises alkyl and/or alkenyl radicals with a number of carbon atoms of 12. Analogously, the term “C14 radical” includes alkyl and/or alkenyl radicals with a number of carbon atoms of 14.

The nanoemulsions according to the invention comprise the alkyl and/or alkenyl ethers or alkyl and/or alkenyl ether mixtures of alkyl and/or alkenyl polyglycosides usually in amounts of from 2 to 25% by weight, in particular 3 to 20% by weight, preferably 5 to 10% by weight, based on the nanoemulsion.

Method of Preparing the Alkyl and/or Alkenyl Ethers of Alkyl and/or Alkenyl (Poly)Glycosides and Mixtures Thereof

The alkyl and/or alkenyl ethers of alkyl and/or alkenyl (poly)glycosides to be used according to the invention can be obtained by reacting alkyl and/or alkenyl (poly)glucosides of formula (II)

Gₘ-R¹ (II)
in which G is a sugar radical having 5 or 6 carbon atoms,
R¹ is a C6 to C22 alkyl and/or alkenyl radical in acetal bond,
m is an average value from 1 to 3, preferably 1.2 to 1.8, and
with alkylating agents of the formula (III) R²—X
   in which X is a nucleophilic leaving group, and
   R² is a C1 to C4 alkyl and/or alkenyl group.

The preferred alkyl and/or alkylene ether mixtures of alkyl and/or alkenyl (poly)glycosides can be obtained by reacting alkyl and/or alkenyl (poly)glucosides of the formula (II)

Gₘ-R¹ (II)
in which G is a sugar radical having 5 or 6 carbon atoms,
R¹ is a C6 to C22 alkyl and/or alkenyl radical in acetal bond,
m is an average value from 1 to 3, preferably 1.2 to 1.8, and
where at least 50% by weight of the alkyl and/or alkenyl polyglycosides comprise a radical R¹ with a carbon chain greater than or equal to 12
with alkylating agents of the formula (III) R²—X
   in which X is a nucleophilic leaving group, and
   R² is a C1 to C4 alkyl and/or alkenyl group.

The invention further provides a method of preparing alkyl and/or alkenyl ether mixtures of the general formula (I-C), characterized in that alkyl and/or alkenyl polyglycosides of the formula (II)

Gluₘ-R¹ (II)
in which G is a sugar radical having 5 or 6 carbon atoms,
R¹ is a C6 to C22 alkyl and/or alkenyl radical in acetal bond,
m is an average value from 1.2 to 1.8, and
where at least 50% by weight of the alkyl and/or alkenyl polyglycosides comprise a radical R¹ with a carbon chain greater than or equal to 12
are reacted with alkylating agents of the formula (III) R²—X
   in which X is a nucleophilic leaving group, and
   R² is a C1 to C4 alkyl and/or alkenyl group.

Suitable starting materials for the alkyl and/or alkenyl ethers of alkyl and/or alkenyl (poly)glycosides according to the invention and mixtures thereof are alkyl and/or alkenyl oligoglycosides of the general formula (II):

Gₘ-R¹ (II)
in which G is a sugar radical having 5 or 6 carbon atoms,
R¹ is a C6 to C22 alkyl and/or alkenyl radical in acetal bond,
m is an average value from 1 to 3, preferably 1.2 to 1.8.

Suitable starting materials for the alkyl and/or alkenyl ethers of alkyl and/or alkenyl (poly)glycosides according to the invention and in particular starting materials for their mixtures are alkyl and/or alkenyl oligoglycosides of the general formula (II):

Gₘ-R¹ (II)
in which G is a sugar radical having 5 or 6 carbon atoms,
R¹ is a C6 to C22 alkyl and/or alkenyl radical in acetal bond,
m is an average value from 1 to 3, preferably 1.2 to 1.8, and
where at least 50% by weight of the alkyl and/or alkenyl polyglycosides comprise a radical R¹ with a carbon chain greater than or equal to 12.

Suitable starting materials for the alkyl and/or alkenyl ethers of alkyl and/or alkenyl (poly)glycosides according to the invention and in particular starting materials for their mixtures are alkyl and/or alkenyl oligoglycosides of the general formula (II):

Gₘ-R¹ (II)
in which G is a sugar radical having 5 or 6 carbon atoms,
R¹ is a C6 to C22 alkyl and/or alkenyl radical in acetal bond,
m is an average value from 1.2 to 1.8, and
where at least 50% by weight of the alkyl and/or alkenyl polyglycosides comprise a radical R¹ with a carbon chain greater than or equal to 12.

These starting materials can be obtained by the relevant methods of preparative organic chemistry. By way of representation of the extensive literature, reference may be made here to the specifications EP-A1-0 301 298 and WO 90/03977.

Suitable starting materials are, for example, the alkyl polyglucosides available under the trade name Plantacare® 1200 from Cognis (INCI Lauryl Glucoside).

The alkyl or alkenyl radical R¹ can be derived from primary alcohols having 8 to 10 carbon atoms. Typical examples are butanol, caproic alcohol, capryl alcohol, capric alcohol and undecyl alcohol and also technical-grade mixtures thereof, as are obtained, for example, in the hydrogenation of technical-grade fatty acid methyl esters or in the course of the hydrogenation of aldehydes from the Roelen oxo synthesis. The alkyl or alkenyl radical R¹ can in addition also be derived from primary alcohols having 12 to 22, preferably 12 to 16, carbon atoms. Typical examples are lauryl alcohol, isotridecyl alcohol, myristyl alcohol, cetyl alcohol, isocetyl alcohol, palmoleyl alcohol, stearyl alcohol, isostearyl alcohol, ricinol alcohol, hydroxystearyl alcohol, dihydroxystearyl alcohol, oleyl alcohol, elaidyl alcohol, petroselinyl alcohol, arachyl alcohol, gadoleyl alcohol, behenyl alcohol, erucyl alcohol, brassidyl alcohol, and technical-grade mixtures thereof, which can be obtained as described above. Guerbet alcohols having 12 to 36 carbon atoms and also technical-grade dimerdiol and trimertriol mixtures having 18 to 36 or 18 to 54 carbon atoms can likewise be used.

### Alkylating and Alkenylating Agents

Alkylating and alkenylating agents which can be used are all compounds which permit an etherification of the free OH groups of the alkyl and/or alkenyl polyglycosides. Examples which may be mentioned are compounds of the type R²—X, where X is a nucleophilic leaving group. R² is the alkyl and/or alkenyl group which is linked via the oxygen group of the alkyl and/or alkenyl polyglycoside in ether bond. R² is a C1 to C4 alkyl and/or alkenyl group. This may be linear or branched, examples which may be mentioned being methyl, ethyl, propyl, isopropyl, n-butyl, 2-methylpropyl (=isobutyl), 1-methylpropyl (=sec-butyl) and 1,1-dimethylethyl (=tert-butyl), ethenyl (=vinyl), propen-1-enyl, propen-2-enyl, isopropenyl.

Examples of suitable alkylating and alkenylating agents of the type R²—X are alkyl and alkenyl halides and/or alkyl and alkenyl tosylates and/or dialkyl and dialkenyl sulfates.

Alkyl tosylates (=alkyl p-toluenesulfonates) which may be mentioned by way of example are methyl tosylate, ethyl tosylate and benzyl tosylate. Alkyl sulfates which may be mentioned by way of example are dimethyl sulfate, diethyl sulfate, dipropyl sulfate and dibutyl sulfate.

Particularly preferred alkylating agents are alkyl halides, in particular chlorides and/or iodides. In one preferred embodiment of the invention, the alkylating agents are selected from the group consisting of methyl chloride, ethyl chloride, n-propyl chloride, isopropyl chloride, n-butyl chloride, isobutyl chloride, sec-butyl chloride, and tert-butyl chloride, and also the corresponding bromine and iodine compounds.

It is also possible to use mixtures of the alkylating and alkenylating agents.

### Alkylation and Alkenylation Reaction

The alkyl and/or alkenyl ethers of alkyl and/or alkenyl (poly)glycosides according to the invention and their mixtures can be obtained by reacting alkyl and/or alkenyl polyglycosides with alkylating or alkenylating agents.

The alkylation or alkenylation reaction is usually carried out at 20 to 100° C., in particular at 40 to 90° C., preferably at 60 to 90° C. The reaction is usually carried out at increased pressure, i.e. at 2 to 10 bar, preferably at 3 to 5 bar. Suitable solvents are water or mixtures of water with low molecular weight alcohols, such as, for example, isopropanol or 1,2-propylene glycol.

The reaction is usually maintained in the presence of alkaline compounds, for example alkali metal hydroxides, such as sodium hydroxide or potassium hydroxide. It has proven advantageous to use the alkali metal hydroxides in equimolar amounts or in molar excess, based on the alkylating or alkenylating agent.

The products according to the invention can be obtained by reacting alkyl and/or alkenyl polyglycosides with the alkylating or alkenylating agents, for example in the molar ratio from 1:3 to 1:10, in particular from 1:3 to 1:8.

In a preferred embodiment of the invention, the ethers and ether mixtures are desalted after the reaction. The desalting can be carried out, for example, by freeze-drying with downstream, if appropriate, repeated, extraction with ethanol. The desalting can also be carried out using customary membrane methods, such as, for example, ultrafiltration or diafiltration.

The invention further provides a method of preparing alkyl and/or alkenyl ether mixtures of the general formula (I-C), characterized in that alkyl and/or alkenyl polyglycosides of the formula (II)

Gluₘ-R¹ (II)
in which G is a sugar radical having 5 or 6 carbon atoms,
R¹ is a C6 to C22 alkyl and/or alkenyl radical in acetal bond,
m is an average value from 1.2 to 1.8, and
where at least 50% by weight of the alkyl and/or alkenyl polyglycosides comprise a radical R¹ with a carbon chain greater than or equal to 12
are reacted with alkylating agents of the formula (III) R²—X
   in which X is a nucleophilic leaving group, and
   R² is a C1 to C4 alkyl and/or alkenyl group,
      and the alkyl and/or alkenyl ether mixture obtained in this way is then desalted.

### Preparation of Nanoemulsions

Various methods are available to the person skilled in the art for preparing nanoemulsions. Besides the dilution of microemulsions and the high-pressure homogenization, the phase inversion temperature method (so-called PIT method) is the most often used method. However, the PIT method has hitherto been limited to systems which mandatorily comprise an ethoxylated emulsifier.

Surprisingly, it has been found that stable nanoemulsions are obtained using the alkyl and/or alkenyl ethers of alkyl and/or alkenyl (poly)glycosides of the formula (I-A) and/or (I-B).

The invention therefore further provides the use of alkyl and/or alkenyl ethers of alkyl and/or alkenyl (poly)glycosides of the formula (I-A)

(Gₘ-R¹)R²ₙ (I-A)
in which G is a sugar radical having 5 or 6 carbon atoms,
R¹ is a C6 to C22 alkyl and/or alkenyl radical in acetal bond,
R² is a C1 to C4 alkyl and/or alkenyl group in ether bond,
m is an average value from 1.0 to 3.0, preferably 1.2 to 1.8, and
n is a number from 0.5 to 5.0, preferably 1.4 to 2.6
and/or of alkyl and/or alkenyl ether mixtures of alkyl and/or alkenyl (poly)glycosides of the formula (I-B)

(Gₘ-R¹)R²ₙ (I-B)
in which G is a sugar radical having 5 or 6 carbon atoms,
R¹ is a C6 to C22 alkyl and/or alkenyl radical in acetal bond,
R² is a C1 to C4 alkyl and/or alkenyl group in ether bond,
m is an average value from 1.0 to 3.0, preferably 1.2 to 1.8, and
n is a number from 0.5 to 5.0, preferably 1.4 to 2.6,
where at least 50% by weight of the alkyl and/or alkenyl ethers comprise a radical R² with a carbon chain greater than or equal to 12,
   for the preparation of nanoemulsions, in particular for the preparation of nanoemulsions according to the PIT method.

The invention further provides a method of preparing nanoemulsions characterized in that either
the oil phase is heated together with at least one alkyl and/or alkenyl ether of alkyl and/or alkenyl (poly)glycosides of the formula (I-A) and/or (I-B) and/or (I-C) and the water phase to a temperature above the phase inversion temperature and this mixture is cooled or
the oil phase is heated together with at least one alkyl and/or alkenyl ether of alkyl and/or alkenyl (poly)glycosides of the formula (I-A) and/or (I-B) and/or (I-C) to a temperature above the phase inversion temperature, and this is cooled by adding the nonheated water phase.

The phase inversion temperature for the method according to the invention is usually in the range from 15 to 90° C. In the phase inversion temperature range, there is a large leap in the conductivity of the mixture. Consequently, the phase inversion temperature for a given system can be determined easily by conductivity measurement, as described, for example, by P. Izquierdo et al. (Journal of colloid and Interface Science 285, 2005, pp. 388-394) on p. 389 under point 2.2.

According to the invention, preference is given to cooling at a cooling rate of greater than 0.2° C. per minute, preferably greater than 0.5° C. per minute, in particular greater than 0.7° C. per minute, preferably greater than 1° C. per minute.

Besides the oil phase and the alkyl and/or alkenyl ethers of alkyl and/or alkenyl (poly)glycosides of the formula (I), the lipophilic phase can comprise further oil-soluble compounds, such as, for example, the coemulsifiers, UV photoprotective filters, perfume oils etc.

Besides water, the water phase can comprise water-soluble compounds, such as, for example, water-soluble vitamins or water-soluble active ingredients.

### Use of the Nanoemulsions

The nanoemulsions according to the invention are suitable as cosmetic and/or pharmaceutical preparations or as bases for the preparation of cosmetic and/or pharmaceutical preparations. These preparations can be present in the form of aqueous solutions, oils, emulsions (W/O or O/W), creams, lotions, etc.

Alkyl and/or Alkenyl Ether Mixtures of Alkyl and/or Alkenyl Polyglycosides of the Formula (I-C)

The invention further provides alkyl and/or alkenyl ether mixtures of alkyl and/or alkenyl polyglycosides of the formula (I-C)

(Gₘ-R¹)R²ₙ (I-C)
in which G is a sugar radical having 5 or 6 carbon atoms,
R¹ is a C6 to C22 alkyl and/or alkenyl radical in acetal bond,
R² is a C1 to C4 alkyl and/or alkenyl group in ether bond,
m is an average value from 1.2 to 1.8, and
n is a number from 1.4 to 2.6,
where at least 50% by weight of the alkyl and/or alkenyl ethers comprise a radical R² with a carbon chain greater than or equal to 12.

The alkyl and/or alkenyl ether mixtures of the formula (I-C) according to the invention are characterized in that at least 50% by weight of the alkyl and/or alkenyl ethers comprise a radical R¹ with a carbon chain greater than or equal to 12. In a preferred embodiment of the invention, more than 50% by weight of the ethers (=alkyl and/or alkenyl ethers of alkyl and/or alkenyl polyglycosides) carry a radical R¹ with a carbon chain greater than or equal to 12, preferably more than 60% by weight, in particular more than 70% by weight, preferably more than 75% by weight. The % by weight refer to the total amount of the alkyl and/or alkenyl ether mixture.

In one embodiment of the invention, the ether mixtures of the formula (I-C) according to the invention comprise ethers where R¹=C12 (=C12 radical) and R¹=C14 (=C14 radical), and the sum of the alkyl and/or alkenyl ethers where R¹=C12 and C14 constitutes more than 50% by weight, in particular more than 60% by weight, preferably more than 70% by weight, in particular more than 75% by weight, preferably more than 80% by weight, based on the total amount of the alkyl and/or alkenyl ether mixture.

The term “C12 radical” includes alkyl and/or alkenyl radicals with a number of carbon atoms of 12. Analogously, the term “C14 radical” includes alkyl and/or alkenyl radicals with a number of carbon atoms of 14.

In a preferred embodiment of the invention, the alkyl and/or alkenyl ether mixtures of the formula (I-C) according to the invention comprise less than 15% by weight, in particular less than 10% by weight, preferably less than 5% by weight, particularly preferably less than 2% by weight, of salt, in particular NaCl. The % by weight are based on the sum of the alkyl and/or alkenyl ether mixtures of the formula (I-C) according to the invention.

The ether mixtures of the formula (I-C) according to the invention are suitable for or for preparing cosmetic and/or pharmaceutical preparations. In this connection they are used, for example, as emulsifiers, preferably as solubilizers.

The ether mixtures of the formula (I-C) according to the invention are suitable in particular for or for preparing cosmetic and/or pharmaceutical preparations in the form of nanoemulsions.

The ether mixtures of the formula (I-C) according to the invention are particularly suitable as solubilizers. They are suitable in particular for solubilizing lipophilic substances, such as, for example, lipophilic cosmetic or lipophilic pharmaceutical active ingredients.

Also provided are preparations comprising 0.1 to 20% by weight of an ether mixture of the formula (I-C). The % by weight are based on the total weight of the preparation. These preparations may be, for example, cosmetic preparations or bases for preparing pharmaceutical preparations. These preparations can be present in the form of aqueous solutions, oils, emulsions (W/O or O/W), creams, lotions, etc.

### Preparations and Nanoemulsions

The preparations according to the invention and also the nanoemulsions according to the invention are suitable in particular also for light, sprayable applications and/or as constituents of care emulsions for tissues, papers, wipes, sponges (e.g. polyurethane sponges), plasters in the sector of baby hygiene, baby care, skincare, sun protection, aftersun treatment, insect repellent, cleansing, facial cleansing and antiperspirant/deodorant application. They can be applied to tissues, papers, wipes, nonwoven products, sponges, puffs, plasters and bandages which are used in the field of cleansing, hygiene and/or care (wet wipes for baby hygiene and baby care, cleansing wipes, facial cleansing wipes, skincare wipes, care wipes with active ingredients to combat skin aging, wipes with sunscreen formulations and insect repellents and also wipes for decorative cosmetics or for aftersun treatment, toilet wet wipes, antiperspirant wipes, diapers, tissues, wet wipes, hygiene products, self-tanning wipes, toilet paper, refreshing wipes, aftershave wipes). They can be used inter alia also in preparations for haircare, hair cleaning or hair coloring. They are suitable in particular as constituents of preparations of decorative cosmetics, such as, for example, lipsticks, eye make-up, such as, for example, eye shadow, mascara, eye pencils, kohl, nail varnish, etc. and also make-up formulations.

### Coemulsifiers

In a preferred embodiment of the invention, the nanoemulsions comprise at least one further emulsifier (=coemulsifier).

The invention further provides preparations comprising alkyl and/or alkenyl ether mixture of alkyl and/or alkenyl polyglycosides of the formula (I-C)

(Gₘ-R¹)R²ₙ (I-C)
in which G is a sugar radical having 5 or 6 carbon atoms,
R¹ is a C6 to C22 alkyl and/or alkenyl radical in acetal bond,
R² is a C1 to C4 alkyl and/or alkenyl group in ether bond,
m is an average value from 1.2 to 1.8, and
n is a number from 1.4 to 2.6,
where at least 50% by weight of the alkyl and/or alkenyl ethers comprise a radical R¹ with a carbon chain greater than or equal to 12,
   and at least one emulsifier (=coemulsifier).

The nanoemulsions according to the invention and the preparations according to the invention comprise the coemulsifier(s) usually in an amount of from 0.01 to 40% by weight, preferably 0.05 to 30% by weight, in particular 0.05 to 20% by weight, preferably 0.1 to 15% by weight and in particular 0.1 to 10% by weight, in particular 0.5 to 5% by weight, based on the nanoemulsion or on the preparation.

In a preferred embodiment of the invention, the weight ratio of coemulsifiers to the alkyl and/or alkenyl ether mixture of alkyl and/or alkenyl (poly)glycosides is 0 to 1.0, preferably 0 to 0.7, in particular 0 to 0.5.

A suitable coemulsifier is in principle any surface-active substance, but in particular substances with an HLB value of from 1 to 20 according to the Griffin scale. Each emulsifier is assigned a so-called HLB value (a dimensionless number between 1 and 20, Griffin scale) which indicates whether a preferred solubility in water or oil is present. Numbers below 9 indicate preferably oil-soluble, hydrophobic emulsifiers; numbers above 11 water-soluble, hydrophilic emulsifiers. The HLB value says something about the equilibrium of the size and strength of the hydrophilic and of the lipophilic groups in an emulsifier. The Griffin scale is described in W C Griffin, J. Soc. Cosmet. Chem. 1 (1949) 311; W C Griffin, J. Soc. Cosmet. Chem. 5 (1954) 249.

The HLB value of an emulsifier can also be calculated from increments, where the HLB increments for the various hydrophilic and hydrophobic groups from which a molecule is composed can be found in tables (e.g. H. P. Fiedler, Lexikon der Hilfsstoffe für Pharmazie, Kosmetik and angrenzende Gebiete [Lexicon of Auxiliaries for Pharmacy, Cosmetics and Related Fields], Editio Cantor Verlag, Aulendorf, 4^{th} edition, 1996) or the manufacturers' information. The solubility of the emulsifier in the two phases practically determines the type of emulsion. If the emulsifier is more soluble in water, then an O/W emulsion is obtained. By contrast, if the emulsifier has better solubility in the oil phase, then under otherwise identical preparation conditions, a W/O emulsion is formed.

In one embodiment of the invention, the preparation according to the invention comprises more than one coemulsifier. Depending on the other components, the person skilled in the art uses customary emulsifier systems (such as, for example, emulsifier and coemulsifier).

According to the invention, preferred coemulsifiers are compounds with an HLB value of less than or equal to 11, preferably less than or equal to 10. Suitable coemulsifiers are in particular compounds with an HLB value of from 1 to 9.

In a preferred embodiment of the invention, the nanoemulsions or the preparations comprise less than 10% by weight, preferably less than 5% by weight, in particular less than 2% by weight, especially less than 0.5% by weight of ethoxylated emulsifiers.

For this reason, of the nonionic emulsifiers specified below, in particular the nonethoxylated representatives of groups (3) and (4), and also the representatives of group (6) and also (8), (9) and (10) are suitable.

### Nonionic Emulsifiers

The group of nonionic emulsifiers includes, for example:
(1) Addition products of from 0 to 50, in particular from 2 to 50, mol of ethylene oxide and/or 0 to 20, in particular from 1 to 20, mol of propylene oxide onto linear and/or branched fatty alcohols having 8 to 40 carbon atoms, onto fatty acids having 12 to 40 carbon atoms and onto alkylphenols having 8 to 15 carbon atoms in the alkyl group.
(2) C₁₂-C₁₈ fatty acid mono- and diesters of addition products of from 1 to 50 mol of ethylene oxide onto glycerol.
(3) Sorbitan mono- and diesters of saturated and unsaturated fatty acids having 6 to 22 carbon atoms and ethylene oxide addition products thereof.
(4) Alkyl mono- and oligoglycosides having 8 to 22 carbon atoms in the alkyl radical and ethoxylated analogs thereof.
(5) Addition products of from 7 to 60 mol of ethylene oxide onto castor oil and/or hydrogenated castor oil.
(6) Polyol and in particular polyglycerol esters, such as, for example, polyol poly-12-hydroxystearates, polyglycerol polyricinoleate, polyglycerol diisostearate or polyglycerol dimerate. Likewise suitable are mixtures of compounds of two or more of these substance classes.
(7) Addition products of from 2 to 15 mol of ethylene oxide onto castor oil and/or hydrogenated castor oil.
(8) Partial esters based on linear, branched, unsaturated or saturated C₆-C₂₂-fatty acids, ricinoleic acid and 12-hydroxystearic acid and polyglycerol, pentaerythritol, dipentaerythritol, sugar alcohols (e.g. sorbitol), alkyl glucosides (e.g. methyl glucoside, butyl glucoside, lauryl glucoside) and polyglucosides (e.g. cellulose), or mixed esters such as, for example, glyceryl stearate citrate and glyceryl stearate lactate.
(9) Polysiloxane-polyalkyl-polyether copolymers and corresponding derivatives.
(10) Mixed esters of pentaerythritol, fatty acids, citric acid and fatty alcohol and/or mixed esters of fatty acids having 6 to 22 carbon atoms, methylglucose and polyols, preferably glycerol or polyglycerol.

The addition products of ethylene oxide and/or of propylene oxide onto fatty alcohols, fatty acids, alkylphenols, glycerol mono- and diesters and also sorbitan mono- and diesters of fatty acids and onto castor oil are known, commercially available products. These are homolog mixtures whose average degree of alkoxylation corresponds to the ratio of the quantitative amounts of ethylene oxide and/or propylene oxide and substrate with which the addition reaction is carried out. Depending on the degree of ethoxylation, they are W/O or O/W emulsifiers. C_{12/18}-fatty acid mono- and diesters of addition products of ethylene oxide onto glycerol are known as refatting agents for cosmetic preparations.

Particularly highly suitable and mild emulsifiers according to the invention are polyol poly-12-hydroxy-stearates and mixtures thereof, which are sold, for example, under the trade names “Dehymuls® PGPH” (W/O emulsifier) or “Eumulgin® VL 75” (mixture with Lauryl Glucosides in the weight ratio 1:1, O/W emulsifier) or Dehymuls® SBL (W/O emulsifier) by Cognis Deutschland GmbH. In this connection, reference may be made in particular to the European patent EP 766 661 B1. The polyol component of these emulsifiers can be derived from substances which have at least two, preferably 3 to 12 and in particular 3 to 8, hydroxyl groups and 2 to 12 carbon atoms.

Particularly preferred emulsifiers are, for example, Cetyl Dimethicone Copolyol (e.g. Abil EM-90), Polyglyceryl-2 Dipolyhydroxystearate (e.g. Dehymuls PGPH), Polyglycerin-3-Diisostearate (e.g. Lameform TGI), Polyglyceryl-4 Isostearate (e.g. Isolan GI 34), Polyglyceryl-3 Oleate (e.g. Isolan GO 33), Glyceryl Stearate Citrate (e.g. Axol C 62, Imwitor 370 and 372P, Dracorin CE 614035), Diisostearoyl Polyglyceryl-3 Diisostearate (e.g. Isolan PDI), Polyglyceryl-3 Methylglucose Distearate (e.g. Tego Care 450), Polyglyceryl-3 Beeswax (e.g. Cera Bellina), Polyglyceryl-4 Caprate (e.g. Polyglycerol Caprate T2010/90), Polyglyceryl-3 Cetyl Ether (e.g. Chimexane NL), Polyglyceryl-3 Distearate (e.g. Cremophor GS 32) and Polyglyceryl Polyricinoleate (e.g. Admul WOL 1403), Glyceryl Oleate (e.g. Monomuls 90-O 18), Alkyl Glucoside (e.g. Plantacare 1200, Emulgade PL 68/50, Montanov 68, Tego Care CG 90, Tego Glucosid L 55), Methyl Glucose Isostearate (e.g. Tego Care IS), Methyl Glucose Sesquistearate (Tego Care PS), Sodium Cocoyl Hydrolyzed Wheat Protein (e.g. Gluadin WK), Potassium Cetyl Phosphate (e.g. Amphisol K, Crodafos CKP), Sodium Alkylsulfate (e.g. Lanette E), Sucrose Ester (e.g. Crodesta F-10, F-20, F-50, F-70, F-110, F-160, SL-40, Emulgade® Sucro), ethoxylated and/or propoxylated fatty alcohols, castor oils and hydrogenated castor oils (e.g. Eumulgin B2, B2, B3, L, HRE 40, HRE 60, RO 40, Cremophor HRE 40, HRE 60, L, WO 7, Dehymuls HRE 7, Arlacel 989), PEG-30 Dipolyhydroxystearate (e.g. Arlacel P 135, Dehymuls LE), sorbitan esters, sorbitan esters ethoxylated and/or propoxylated and mixtures thereof. A particularly effective mixture consists of Polyglyceryl-2 Dipolyhydroxystearate and Lauryl Glucoside and Glycerol (e.g. Eumulgin VL 75). Also suitable are Polyglyceryl-4 Diisostearate-/Polyhydroxystearate/Sebacate (Isolan® GPS), Diisostearoyl Polyglyceryl-3 Diisostearate (e.g. Isolan PDI), alkali metal salts Acylglutamate (e.g. Eumulgin SG).

Of suitability in principle as lipophilic W/O emulsifiers are emulsifiers with an HLB value of from 1 to 8 which are summarized in numerous tables and are known to the person skilled in the art. Some of these emulsifiers are listed, for example, in Kirk-Othmer, “Encyclopedia of Chemical Technology”, 3^{rd} edition, 1979, volume 8, page 913. For ethoxylated products, the HLB value can also be calculated according to the following formula: HLB=(100−L):5, where L is the weight fraction of the lipophilic groups, i.e. of the fatty alkyl or fatty acyl groups in weight percent, in the ethylene oxide adducts.

Of particular advantage from the group of W/O emulsifiers are partial esters of polyols, in particular of C₄-C₆-polyols, such as, for example, partial esters of pentaerythritol or sugar esters, e.g. sucrose distearate, sorbitan monoisostearate, sorbitan sesquiisostearate, sorbitan diisostearate, sorbitan triisostearate, sorbitan monooleate, sorbitan sesquioleate, sorbitan dioleate, sorbitan trioleate, sorbitan monoerucate, sorbitan sesquierucate, sorbitan dierucate, sorbitan trierucate, sorbitan monoricinoleate, sorbitan sesquiricinoleate, sorbitan diricinoleate, sorbitan triricinoleate, sorbitan monohydroxystearate, sorbitan sesquihydroxystearate, sorbitan dihydroxystearate, sorbitan trihydroxy-stearate, sorbitan monotartrate, sorbitan sesquitartrate, sorbitan ditartrate, sorbitan tritartrate, sorbitan monocitrate, sorbitan sesquicitrate, sorbitan dicitrate, sorbitan tricitrate, sorbitan monomaleate, sorbitan sesquimaleate, sorbitan dimaleate, sorbitan trimaleate and technical-grade mixtures thereof. Also suitable as emulsifiers are addition products of from 1 to 30, preferably 5 to 10, mol of ethylene oxide onto the specified sorbitan esters.

Depending on the formulation, it may be advantageous to additionally use at least one emulsifier from the group of nonionic O/W emulsifiers (HLB value: 8-18) and/or solubilizers. These are, for example, the ethylene oxide adducts already mentioned in the introduction and having a correspondingly high degree of ethoxylation, e.g. 10-20 ethylene oxide units for O/W emulsifiers and 20-40 ethylene oxide units for so-called solubilizers. According to the invention, Ceteareth-12 and PEG-20 Stearate are particularly advantageous as O/W emulsifiers. Suitable solubilizers are preferably Eumulgin® HRE 40 (INCI: PEG-40 Hydrogenated Castor Oil), Eumulgin® HRE 60 (INCI: PEG-60 Hydrogenated Castor Oil), Eumulgin® L (INCI: PPG-1-PEG-9 Lauryl Glycol Ether), and Eumulgin® SML 20 (INCI: Polysorbate-20).

Nonionic emulsifiers from the group of alkyl oligoglycosides are particularly skin-friendly and therefore preferably suitable as O/W emulsifiers. C₈-C₂₂-alkyl mono and oligoglycosides, their preparation and their use are known from the prior art. Their preparation takes place in particular by reacting glucose or oligosaccharides with primary alcohols having 8 to 22 carbon atoms. As regards the glycoside radical, either monoglycosides, in which a cyclic sugar radical is glycosidically bonded to the fatty alcohol, or oligomeric glycosides with a degree of oligomerization up to preferably about 8 are suitable. The degree of oligomerization here is a statistical average value based on a homolog distribution customary for such technical-grade products. Products which are available under the name Plantacare® comprise a glucosidically bonded C₈-C₁₆-alkyl group onto an oligoglucoside radical whose average degree of oligomerization is 1 to 2. The acylglucamides derived from glucamine are also suitable as nonionic emulsifiers. According to the invention, preference is given to a product which is sold under the name Emulgade® PL 68/50 by Cognis Deutschland GmbH and is a 1:1 mixture of alkyl polyglucosides and fatty alcohols. According to the invention, it is also advantageously possible to use a mixture of Lauryl Glucoside, Polyglyceryl-2 Dipolyhydroxystearate, glycerol and water, which is commercially available under the name Eumulgin® VL 75.

Also suitable as emulsifiers are substances such as lecithins and phospholipids. Examples of natural lecithins which may be mentioned are the cephalins, which are also referred to as phosphatidic acids and are derivatives of 1,2-diacyl-sn-glycerol-3-phosphoric acids. By contrast, phospholipids are usually understood as meaning mono- and preferably diesters of phosphoric acid with glycerol (glycerol phosphates), which are generally included in the fats. In addition, sphingosines and sphingolipids are also suitable.

Silicone emulsifiers, for example, may be present as emulsifiers. These can be selected, for example, from the group of alkylmethicone copolyols and/or alkyldimethicone copolyols, in particular from the group of compounds which are characterized by the following chemical structure:

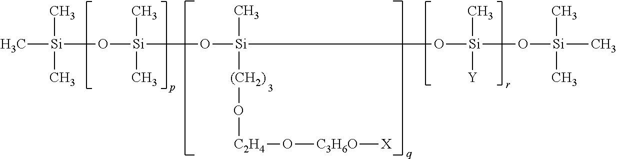

in which X and Y, independently of one another, are selected from the group H (hydrogen) and the branched and unbranched alkyl groups, acyl groups and alkoxy groups having 1-24 carbon atoms, p is a number from 0-200, q is a number from 1-40, and r is a number from 1-100.

One example of silicone emulsifiers to be used particularly advantageously within the context of the present invention are dimethicone copolyols, which are sold by Evonik Goldschmidt under the trade names AXIL® B 8842, ABIL® B 8843, ABIL® B 8847, ABIL® B 8851, ABIL® B 8852, ABIL® B 8863, ABIL® B 8873 and ABIL®B 88183.

A further example of interface-active substances to be used particularly advantageously within the context of the present invention is cetyl PEG/PPG-10/1 dimethicone (Cetyl Dimethiconecopolyol), which is sold by Evonik Goldschmidt under the trade name ABIL® EM 90.

A further example of interface-active substances to be used particularly advantageously within the context of the present invention is the cyclomethicone dimethiconecopolyol, which is sold by Evonik Goldschmidt under the trade name ABIL® EM 97 and ABIL® WE 09.

Furthermore, the emulsifier Lauryl PEG/PPG-18/18 Methicone (laurylmethicone copolyol) has proven to be very particularly advantageous and is available under the trade name Dow Corning® 5200 Formulation Aid from Dow Corning Ltd.

A further advantageous silicone emulsifier is Octyl Dimethicone Ethoxy Glucoside from Wacker. For a water-in-silicone oil emulsion according to the invention, all known emulsifiers used for this type of emulsion can be used. According to the invention, particularly preferred water-in-silicone emulsifiers here are cetyl PEG/PPG-10/1 dimethicones and lauryl PEG/PPG-18/18 methicones [e.g. ABIL® EM 90 (Evonik Goldschmidt), DC5200 Formulation Aid (Dow Corning)] and any desired mixtures of the two emulsifiers.

Surfactants can likewise be used as coemulsifier. Surfactants are amphiphilic substances which can dissolve organic, nonpolar substances in water. As a result of their specific molecular structure with at least one hydrophilic and one hydrophobic molecular moiety, they provide for a reduction in the surface tension of water, wetting of the skin, facilitation of soil removal and release, ease of rinsing off and—if desired for foam regulation.

Coemulsifiers which may be present are anionic, nonionic, cationic and/or amphoteric or zwitterionic surfactants. In surfactant-containing cosmetic preparations, such as, for example, shower gels, foam baths, shampoos etc., preferably at least one anionic surfactant is present.

Typical examples of nonionic surfactants are fatty alcohol polyglycol ethers, alkylphenol polyglycol ethers, fatty acid polyglycol esters, fatty acid amide polyglycol ethers, fatty amine polyglycol ethers, alkoxylated triglycerides, mixed ethers and mixed formals, optionally partially oxidized alk(en)yl oligoglycosides and glucuronic acid derivatives, fatty acid N-alkylglucamides, protein hydrolyzates (in particular wheat-based vegetable products), polyol fatty acid esters, sugar esters, sorbitan esters, polysorbates and amine oxides. If the nonionic surfactants contain polyglycol ether chains, these may have a conventional homolog distribution, but preferably have a narrowed homolog distribution.

Zwitterionic surfactants is the term used to refer to those surface-active compounds which carry at least one quaternary ammonium group and at least one —COO⁽⁻⁾ or —SO₃⁽⁻⁾ group in the molecule. Particularly suitable zwitterionic surfactants are the so-called betaines, such as the N-alkyl-N,N-dimethylammonium glycinates, for example cocoalkyl dimethylammonium glycinate, N-acylaminopropyl-N,N-dimethylammonium glycinates, for example cocoacylaminopropyldimethylammonium glycinate, and 2-alkyl-3-carboxymethyl-3-hydroxyethylimidazoline having in each case 8 to 18 carbon atoms in the alkyl or acyl group, and also cocoacylaminoethyl hydroxy-ethylcarboxymethyl glycinate. A preferred zwitterionic surfactant is the fatty acid amide derivative known under the INCI name Cocamidopropyl Betaine.

Likewise suitable, especially as cosurfactants, are ampholytic surfactants. Ampholytic surfactants are understood as meaning those surface-active compounds which, apart from a C₈-C₁₈-alkyl or acyl group in the molecule, contain at least one free amino group and at least one —COOH or —SO₃H group and are capable of forming internal salts. Examples of suitable ampholytic surfactants are N-alkylglycines, N-alkylpropionic acids, N-alkylaminobutyric acids, N-alkylimino-dipropionic acids, N-hydroxyethyl-N-alkylamidopropyl-glycines, N-alkyltaurines, N-alkylsarcosines, 2-alkyl-aminopropionic acids and alkylaminoacetic acids having in each case about 8 to 18 carbon atoms in the alkyl group. Particularly preferred ampholytic surfactants are N-cocoalkylaminopropionate, cocoacylaminoethyl-aminopropionate and C₁₂₋₁₈-acylsarcosine.

Typical examples of amphoteric and zwitterionic surfactants are alkylbetaines, alkylamidobetaines, aminopropionates, aminoglycinates, imidazolinium betaines and sulfobetaines. The specified surfactants are exclusively known compounds. With regard to the structure and preparation of these substances, reference may be made to relevant review works in this field. Typical examples of particularly suitable mild, i.e. particularly skin-friendly, surfactants are fatty alcohol polyglycol ether sulfates, monoglyceride sulfates, mono- and/or dialkyl sulfosuccinates, fatty acid isethionates, fatty acid sarcosinates, fatty acid taurides, fatty acid glutamates, α-olefinsulfonates, ether carboxylic acids, alkyl oligoglucosides and/or mixtures thereof with alkyl oligoglucoside carboxylates, fatty acid glucamides, alkylamidobetaines, amphoacetals and/or protein fatty acid condensates, the latter preferably based on wheat proteins or salts thereof.

Anionic surfactants are characterized by a water-solubilizing, anionic group, such as, for example, a carboxylate, sulfate, sulfonate or phosphate group and a lipophilic radical. Skin-compatible anionic surfactants are known to the person skilled in the art in a large number from relevant handbooks and are commercially available. These are in particular alkyl sulfates in the form of their alkali metal, ammonium or alkanolammonium salts, alkyl ether sulfates, alkyl ether carboxylates, acyl isethionates, acyl sarcosinates, acyltaurines with linear alkyl or acyl groups having 12 to 18 carbon atoms, and also sulfosuccinates and acyl glutamates in the form of their alkali metal or ammonium salts.

Typical examples of anionic surfactants are soaps, alkylbenzenesulfonates, alkanesulfonates, olefin-sulfonates, alkyl ether sulfonates, glycerol ether sulfonates, α-methyl ester sulfonates, sulfofatty acids, alkyl sulfates, fatty alcohol ether sulfates, glycerol ether sulfates, fatty acid ether sulfates, hydroxy mixed ether sulfates, monoglyceride (ether) sulfates, fatty acid amide (ether) sulfates, mono- and dialkyl sulfosuccinates, mono- and dialkyl sulfosuccinamates, sulfotriglycerides, amide soaps, ethercarboxylic acids and salts thereof, fatty acid isethionates, fatty acid sarcosinates, fatty acid taurides, N-acylamino acids, such as, for example, acyl lactylates, acyl tartrates, acyl glutamates and acyl aspartates, alkyl oligoglucoside sulfates, alkyl oligoglucoside carboxylates, protein fatty acid condensates (in particular vegetable products based on wheat) and alkyl (ether) phosphates. If the anionic surfactants comprise polyglycol ether chains, these may have a conventional homolog distribution, but preferably have a narrowed homolog distribution.

Cationic surfactants which can be used are in particular quaternary ammonium compounds. Preference is given to ammonium halides, in particular chlorides and bromides, such as alkyltrimethylammonium chlorides, dialkyldimethylammonium chlorides and trialkyl-methylammonium chlorides, e.g. cetyltrimethylammonium chloride, stearyltrimethylammonium chloride, distearyl-dimethylammonium chloride, lauryldimethylammonium chloride, lauryldimethylbenzylammonium chloride and tricetylmethylammonium chloride. Furthermore, the very readily biodegradable quaternary ester compounds, such as, for example, the dialkylammonium methosulfates and methylhydroxyalkyldialkoyloxyalkylammonium metho-sulfates sold under the trade name Stepantex® and the corresponding products of the Dehyquart® series can also be used as cationic surfactants. The term “ester quats” are generally understood as meaning quaternized fatty acid triethanolamine ester salts. They can impart a particular soft feel to the preparations according to the invention. These are known substances which are prepared by the relevant methods of organic chemistry. Further cationic surfactants which can be used according to the invention are the quaternized protein hydrolyzates.

### Oil Phase

The nanoemulsions according to the invention comprise an oil phase.

The invention further provides preparations comprising alkyl and/or alkenyl ether mixture of alkyl and/or alkenyl polyglycosides of the formula (I-C)

(Gₘ-R¹)R²ₙ (I-C)
in which G is a sugar radical having 5 or 6 carbon atoms,
R¹ is a C6 to C22 alkyl and/or alkenyl radical in acetal bond,
R² is a C1 to C4 alkyl and/or alkenyl group in ether bond,
m is an average value from 1.2 to 1.8, and
n is a number from 1.4 to 2.6,
where at least 50% by weight of the alkyl and/or alkenyl ethers comprise a radical R² with a carbon chain greater than or equal to 12,
   and at least one oil phase.

The oil phase is usually present in amounts of from 0.1-90, in particular 0.1-80, in particular 0.5 to 70, preferably 1 to 60, in particular 1 to 50% by weight, in particular 1 to 40% by weight, preferably 5-25% by weight and in particular 5-15% by weight, based on the nanoemulsion or based on the preparation.

In one preferred embodiment of the invention, the weight ratio of the sum of all coemulsifiers and of all alkyl and/or alkenyl ethers of alkyl and/or alkenyl (poly)glycosides to the sum of the oil phase is 0.01 to 1.0, preferably 0.05 to 0.75, in particular 0.07 to 0.5.

For the calculation of the oil phase, the alkyl and/or alkenyl ether mixture of alkyl and/or alkenyl (poly)glycosides of the formula (I-A), (I-B) and (I-C) and also the optionally present further emulsifiers are not included in the calculation.

The oil phase can comprise oil bodies, fats and waxes/wax components:

Suitable oil bodies are, for example, Guerbet alcohols based on fatty alcohols having 6 to 18, preferably 8 to 10, carbon atoms, and also further additional esters such as myristyl myristate, myristyl palmitate, myristyl stearate, myristyl isostearate, myristyl oleate, myristyl behenate, myristyl erucate, cetyl myristate, cetyl palmitate, cetyl stearate, cetyl isostearate, cetyl oleate, cetyl behenate, cetyl erucate, stearyl myristate, stearyl palmitate, stearyl stearate, stearyl isostearate, stearyl oleate, stearyl behenate, stearyl erucate, isostearyl myristate, isostearyl palmitate, isostearyl stearate, isostearyl isostearate, isostearyl oleate, isostearyl behenate, isostearyl oleate, oleyl myristate, oleyl palmitate, oleyl stearate, oleyl isostearate, oleyl oleate, oleyl behenate, oleyl erucate, behenyl myristate, behenyl palmitate, behenyl stearate, behenyl isostearate, behenyl oleate, behenyl behenate, behenyl erucate, erucyl myristate, erucyl palmitate, erucyl stearate, erucyl isostearate, erucyl oleate, erucyl behenate and erucyl erucate. Also suitable are esters of C₁₈-C₃₈-alkylhydroxycarboxylic acids with linear or branched C₆-C₂₂-fatty alcohols, in particular dioctyl malate, esters of linear and/or branched fatty acids with polyhydric alcohols (such as, for example, propylene glycol, dimerdiol or trimertriol), triglycerides based on C₆-C₁₀-fatty acids, liquid mono-/di-/triglyceride mixtures based on C₆-C₁₈-fatty acids, esters of C₆-C₂₂-fatty alcohols and/or Guerbet alcohols with aromatic carboxylic acids, in particular benzoic acid, esters of C₂-C₁₂-dicarboxylic acids with polyols having 2 to 10 carbon atoms and 2 to 6 hydroxyl groups, vegetable oils, branched primary alcohols, substituted cyclohexanes, linear and branched C₆-C₂₂-fatty alcohol carbonates, such as, for example, dicaprylyl carbonate (Cetiol® CC), Guerbet carbonates based on fatty alcohols having 6 to 18, preferably 8 to 10, carbon atoms, esters of benzoic acid with linear and/or branched C₆-C₂₂-alcohols (e.g. Finsolv® TN), linear or branched, symmetrical or asymmetrical dialkyl ethers having 6 to 22 carbon atoms per alkyl group, such as, for example, dicaprylyl ether (Cetiol® OE), ring-opening products of epoxidized fatty acid esters with polyols and hydrocarbons or mixtures thereof.

Likewise suitable as oil bodies are esters of linear C6-C22-fatty acids with linear or branched C6-C22-fatty alcohols, in particular esters of C8-fatty acids with 2-propylheptanol, as are available under the trade name Cetiol® SenSoft (INCI: Propylheptyl Caprylate) from Cognis.

Suitable oil bodies are, for example, silicone oils. They may be present as cyclic and/or linear silicone oils. Silicone oils are high molecular weight synthetic polymeric compounds in which silicon atoms are linked via oxygen atoms in a chain-like and/or grid-like manner and the remaining valences of silicon are saturated by hydrocarbon radicals (in most cases methyl, more rarely ethyl, propyl, phenyl groups etc.). Systematically, the silicone oils are referred to as polyorganosiloxanes. The methyl-substituted polyorgano-siloxanes, which are the most important compounds of this group in terms of amount and are characterized by the following structural formula

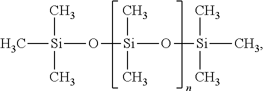

are also referred to as polydimethylsiloxane or dimethicone (INCI). Dimethicones come in various chain lengths and with various molecular weights.

Advantageous polyorganosiloxanes within the context of the present invention are, for example, dimethylpoly-siloxane [poly(dimethylsiloxane)], which are available, for example, under the trade names Abil 10 to 10 000 from Evonik Goldschmidt. Also advantageous are phenylmethylpolysiloxane (INCI: Phenyl Dimethicone, Phenyl Trimethicone), cyclic silicones (octamethyl-cyclotetrasiloxane or decamethylcyclopentasiloxane), which are also referred to in accordance with INCI as cyclomethicone, amino-modified silicones (INCI: Amodimethicones) and silicone waxes, e.g. polysiloxane-polyalkylene copolymers (INCI: Stearyl Dimethicone and Cetyl Dimethicone) and dialkoxydimethylpolysiloxanes (Stearoxy Dimethicone and Behenoxy Stearyl Dimethicone), which are available as various Abil wax grades from Evonik Goldschmidt. However, other silicone oils can also be used advantageously within the context of the present invention, for example cetyldimethicone, hexamethylcyclotrisiloxane, polydimethylsiloxane, poly(methylphenylsiloxane). Silicones that are particularly preferred according to the invention are dimethicone and cyclomethicone.

The term wax/wax component is usually to be understood as meaning all natural or artificially obtained substances and substance mixtures having the following properties: they are from solid to brittly hard consistency, coarse to finely crystalline, transparent to cloudy and melt above 30° C. without decomposition. They are low viscosity even a little above the melting point and are not thread-drawing and exhibit a strongly temperature-dependent consistency and solubility. According to the invention, it is possible to use a wax component or a mixture of wax components which melt at 30° C. or above.

Waxes which can be used according to the invention are also fats and fat-like substances with wax-like consistency provided they have the required melting point. These include, inter alia, fats (triglycerides), mono- and diglycerides, natural and synthetic waxes, fatty and wax alcohols, fatty acids, esters of fatty alcohols and fatty acids and also fatty acid amides or any desired mixtures of these substances.

Fats are understood as meaning triacylglycerols, i.e. the triple esters of fatty acids with glycerol. Preferably, they comprise saturated, unbranched and unsubstituted fatty acid radicals. These may also be mixed esters, i.e. triple esters of glycerol with various fatty acids. According to the invention so-called hydrogenated fats and oils, which are obtained by partial hydrogenation, can be used and are particularly highly suited as consistency regulators. Vegetable hydrogenated fats and oils are preferred, e.g. hydrogenated castor oil, peanut oil, soybean oil, colza oil, rapeseed oil, cottonseed oil, sunflower oil, palm oil, palm kernel oil, linseed oil, almond oil, corn oil, olive oil, sesame oil, cocoa butter and coconut fat.

Inter alia, the triple esters of glycerol with C12-C60-fatty acids and in particular C12-C36-fatty acids are suitable. These include hydrogenated castor oil, a triple ester of glycerol and a hydroxystearic acid, which is commercially available, for example, under the name Cutina HR. Glycerol tristearate, glycerol tribehenate (e.g. Syncrowax HRC), glycerol tripalmitate or the triglyceride mixtures known under the name Syncrowax HGLC are likewise suitable, with the proviso that the melting point of the wax component or of the mixture is 30° C. or above.

According to the invention, wax components which can be used are in particular mono- and diglycerides and mixtures of these partial glycerides. Glyceride mixtures which can be used according to the invention include the products Novata AB and Novata B (mixture of C12-C18-mono-, di- and triglycerides) and Cutina MD or Cutina GMS (glyceryl stearate) marketed by Cognis Deutschland GmbH & Co. KG.

Fatty alcohols which can be used according to the invention as wax component include the C12-C50-fatty alcohols. The fatty alcohols can be obtained from natural fats, oils and waxes, such as, for example, myristyl alcohol, 1-pentadecanol, cetyl alcohol, 1-heptadecanol, stearyl alcohol, 1-nonadecanol, arachidyl alcohol, 1-heneicosanol, behenyl alcohol, brassidyl alcohol, lignoceryl alcohol, ceryl alcohol or myricyl alcohol. According to the invention, preference is given to saturated unbranched fatty alcohols. However, unsaturated, branched or unbranched fatty alcohols can also be used according to the invention as wax component provided they have the required melting point. According to the invention, it is also possible to use fatty alcohol cuts as are produced during the reduction of naturally occurring fats and oils, such as, for example, bovine tallow, peanut oil, colza oil, cottonseed oil, soybean oil, sunflower oil, palm kernel oil, linseed oil, castor oil, corn oil, rapeseed oil, sesame oil, cocoa butter and coconut fat. However, it is also possible to use synthetic alcohols, e.g. the linear, even-numbered fatty alcohols of the Ziegler synthesis (alfols) or the partially branched alcohols from the oxo synthesis (dobanols). According to the invention, C14-C22-fatty alcohols, which are marketed, for example, by Cognis Deutschland GmbH under the name Lanette 18 (C18-alcohol), Lanette 16 (C16-alcohol), Lanette 14 (C14-alcohol), Lanette O (C16/C18-alcohol) and Lanette 22 (C18/C22-alcohol), are particularly preferably suitable. Fatty alcohols give the compositions a dryer skin feel than triglycerides and are therefore preferred over the latter.

Wax components which can be used are also C14-C40-fatty acids or mixtures thereof. These include, for example, myristic acid, pentadecanoic acid, palmitic acid, margaric acid, stearic acid, nonadecanoic acid, arachic acid, behenic acid, lignoceric acid, cerotic acid, melissic acid, erucic acid and elaeostearic acid, and also substituted fatty acids, such as, for example, 12-hydroxystearic acid, and the amides or monoethanolamides of the fatty acids, this list being exemplary and nonlimiting in character.

According to the invention, it is possible to use, for example, natural vegetable waxes, such as candelilla wax, carnauba wax, Japan wax, esparto grass wax, cork wax, guaruma wax, rice germ wax, sugarcane wax, ouricury wax, montan wax, sunflower wax, fruit waxes such as orange waxes, lemon waxes, grapefruit wax, bayberry wax, and animal waxes, such as, for example, beeswax, shellac wax, spermaceti, wool wax and uropygial grease. Within the context of the invention, it may be advantageous to use hydrogenated or hardened waxes. Natural waxes that can be used according to the invention also include mineral waxes, such as, for example, ceresin and ozokerite or the petrochemical waxes, such as, for example, petrolatum, paraffin waxes and microwaxes. Wax components which can be used are also chemically modified waxes, in particular the hard waxes, such as, for example, montan ester waxes, sasol waxes and hydrogenated jojoba waxes. Synthetic waxes which can be used according to the invention include, for example, wax-like polyalkylene waxes and polyethylene glycol waxes. Vegetable waxes are preferred according to the invention.

The wax component can likewise be selected from the group of wax esters of saturated and/or unsaturated, branched and/or unbranched alkanecarboxylic acids and saturated and/or unsaturated, branched and/or unbranched alcohols, from the group of esters of aromatic carboxylic acids, dicarboxylic acids, tricarboxylic acids and hydroxycarboxylic acids (e.g. 12-hydroxystearic acid) and saturated and/or unsaturated, branched and/or unbranched alcohols, and also from the group of lactides of long-chain hydroxycarboxylic acids. Examples of such esters are the C16-C40-alkyl stearates, C20-C40-alkyl stearates (e.g. Kesterwachs K82H), C20-C40-dialkyl esters of dimeric acids, C18-C38-alkylhydroxystearoyl stearates or C20-C40-alkyl erucates. C30-C50-Alkylbeeswax, tristearyl citrate, triisostearyl citrate, stearyl heptanoate, stearyl octanoate, trilauryl citrate, ethylene glycol dipalmitate, ethylene glycol distearate, ethylene glycol di(12-hydroxystearate), stearyl stearate, palmityl stearate, stearyl behenate, cetyl ester, cetearyl behenate and behenyl behenate can also be used. Fatty acid partial glycerides, i.e. technical-grade mono- and/or diesters of glycerol with fatty acids having 12 to 18 carbon atoms, such as, for example, glycerol mono/dilaurate, -palmitate, -myristate or -stearate, are also suitable for this purpose.

Suitable waxes are also pearlescent waxes. Suitable pearlescent waxes, especially for use in surface-active formulations, are, for example: alkylene glycol esters, specifically ethylene glycol distearate; fatty acid alkanolamides, specifically coconut fatty acid diethanolamide; partial glycerides, specifically stearic acid monoglyceride; esters of polybasic, optionally hydroxy-substituted carboxylic acids with fatty alcohols having 6 to 22 carbon atoms, specifically long-chain esters of tartaric acid; fatty substances, such as, for example, fatty alcohols, fatty ketones, fatty aldehydes, fatty ethers and fatty carbonates, which have in total at least 24 carbon atoms, specifically laurone and distearyl ethers; fatty acids such as stearic acid, hydroxystearic acid or behenic acid, ring-opening products of olefin epoxides having 12 to 22 carbon atoms with fatty alcohols having 12 to 22 carbon atoms and/or polyols having 2 to 15 carbon atoms and 2 to 10 hydroxyl groups, and mixtures thereof.

Surprisingly, it has been found that the nanoemulsions according to the invention are particularly suitable for solubilizing oil-soluble UV photoprotective filters.

The invention provides preparations comprising nanoemulsions as claimed in claim 1 and at least one UV photoprotective filter, preferably an oil-soluble UV photoprotective filter.

Surprisingly, it has been found that the ether mixtures of the formula (I-C) according to the invention are particularly suitable for solubilizing oil-soluble UV photoprotective filters.

The invention provides preparations comprising alkyl and/or alkenyl ether mixture of alkyl and/or alkenyl polyglycosides of the formula (I-C)

(Gₘ-R¹)R²ₙ (I-C)
in which G is a sugar radical having 5 or 6 carbon atoms,
R¹ is a C6 to C22 alkyl and/or alkenyl radical in acetal bond,
R² is a C1 to C4 alkyl and/or alkenyl group in ether bond,
m is an average value from 1.2 to 1.8, and
n is a number from 1.4 to 2.6,
   where at least 50% by weight of the alkyl and/or alkenyl ethers comprise a radical R¹ with a carbon chain greater than or equal to 12, and at least one UV photoprotective filter, preferably an oil-soluble UV photoprotective filter.

According to the invention, suitable UV photoprotective filters are organic substances (photoprotective filters) that are crystalline or liquid at room temperature and which are able to absorb ultraviolet rays and release the absorbed energy again in the form of longer-wave radiation, e.g. heat. UV filters may be oil-soluble or water-soluble. Typical oil-soluble UV-B filters and broadband UV A/B filters to be mentioned are, for example:
3-benzylidenecamphor or 3-benzylidenenorcamphor (Mexoryl SDS 20) and derivatives thereof, e.g. 3-(4-methylbenzylidene)camphor as described in EP 0693471 B1
3-(4′-trimethylammonium)benzylidenebornan-2-one methyl sulfate (Mexoryl SO)
3,3′-(1,4-phenylenedimethine)bis(7,7-dimethyl-2-oxobicyclo[2.2.1]heptane-1-methanesulfonic acid) and salts (Mexoryl SX)
3-(4′-sulfo)benzylidenebornan-2-one and salts (Mexoryl SL)
polymer of N-{(2 and 4)-[2-oxoborn-3-ylidene)-methyl}benzyl]acrylamide (Mexoryl SW)
2-(2H-benzotriazol-2-yl)-4-methyl-6-(2-methyl-3-(1,3,3,3-tetramethyl-1-(trimethylsilyloxy)-disiloxanyl)propyl)phenol (Mexoryl XL)
4-aminobenzoic acid derivatives, preferably 2-ethylhexyl 4-(dimethylamino)benzoate, 2-octyl 4-(dimethylamino)benzoate and amyl 4-(dimethyl-amino)benzoate;
esters of cinnamic acid, preferably 2-ethylhexyl 4-methoxycinnamate, propyl 4-methoxycinnamate, isoamyl 4-methoxycinnamate, 2-ethylhexyl 2-cyano-3,3-phenylcinnamate (octocrylene);
esters of salicylic acid, preferably 2-ethylhexyl salicylate, 4-isopropylbenzyl salicylate, homo-menthyl salicylate;
derivatives of benzophenone, preferably 2-hydroxy-4-methoxybenzophenone, 2-hydroxy-4-methoxy-4′-methylbenzophenone, 2,2′-dihydroxy-4-methoxybenzophenone;
esters of benzalmalonic acid, preferably di-2-ethylhexyl 4-methoxybenzmalonate;
triazine derivatives, such as, for example, 2,4,6-trianilino(p-carbo-2′-ethyl-1′-hexyloxy)-1,3,5-triazine and 2,4,6-tris[p-(2-ethylhexyloxy-carbonyl)anilino]-1,3,5-triazine (Uvinul T 150) as described in EP 0818450 A1 or bis(2-ethylhexyl) 4,4′-[(6-[4-((1,1-dimethylethyl)aminocarbonyl)-phenylamino]-1,3,5-triazine-2,4-diyl)diimino]bis-benzoate (Uvasorb® HEB);
2,2-(methylenebis(6-(2H-benzotriazol-2-yl)-4-(1,1,3,3-tetramethylbutyl)phenol) (Tinosorb M);
2,4-bis[4-(2-ethylhexyloxy)-2-hydroxyphenyl]-6-(4-methoxyphenyl)-1,3,5-triazine (Tinosorb S);
propane-1,3-diones, such as, for example, 1-(4-tert-butylphenyl)-3-(4′-methoxyphenyl)propane-1,3-dione;
ketotricyclo(5.2.1.0)decane derivatives, as described in EP 0694521 B1;
dimethicodiethyl benzalmalonates (Parsol SLX).

Suitable water-soluble UV filters are:
2-phenylbenzimidazole-5-sulfonic acid and the alkali metal, alkaline earth metal, ammonium, alkylammonium, alkanolammonium and glucammonium salts thereof;
2,2-((1,4-phenylene)bis(1H-benzimidazole-4,6-disulfonic acid, monosodium salt) (Neo Heliopan AP)
sulfonic acid derivatives of benzophenones, preferably 2-hydroxy-4-methoxybenzophenone-5-sulfonic acid and its salts;
sulfonic acid derivatives of 3-benzylidenecamphor, such as, for example, 4-(2-oxo-3-bornylidenemethyl)benzenesulfonic acid and 2-methyl-5-(2-oxo-3-bornylidene)sulfonic acid and salts thereof.

In one preferred embodiment of the invention, the nanoemulsions or the preparations according to the invention comprise at least one oil-soluble UV photoprotective filter and at least one water-soluble UV photoprotective filter.

Suitable typical UV-A filters are in particular derivatives of benzoylmethane, such as, for example, 1-(4′-tert-butylphenyl)-3-(4′-methoxyphenyl)propane-1,3-dione, 4-tert-butyl-4′-methoxydibenzoylmethane (Parsol® 1789), 1-phenyl-3-(4′-isopropylphenyl)propane-1,3-dione, and enamine compounds, as described in DE 19712033 A1 (BASF) and benzoic acid, 2-[4-(diethyl-amino)-2-hydroxybenzoyl], hexyl ester (Uvinul® A plus).

The UV-A and UV-B filters can of course also be used in mixtures. Particularly favorable combinations consist of the derivatives of benzoylmethane, e.g. 4-tert-butyl-4′-methoxydibenzoylmethane (Parsol® 1789) and 2-ethylhexyl 2-cyano-3,3-phenylcinnamate (octocrylene) in combination with esters of cinnamic acid, preferably 2-ethylhexyl 4-methoxycinnamate and/or propyl 4-methoxycinnamate and/or isoamyl 4-methoxycinnamate. Combinations of this type are advantageously combined with water-soluble filters such as, for example, 2-phenylbenzimidazole-5-sulfonic acid and alkali metal, alkaline earth metal, ammonium, alkylammonium, alkanolammonium and glucammonium salts thereof.

Suitable UV photoprotective filters are in particular the substances approved according to Annex VII of the Commission Directive (in the version Commission Directive 2005/9/EC of 28 Jan. 2005 amending Council Directive 76/768/EEC, concerning cosmetic products, for the purposes of adapting Annexes VII thereof to technical progress), to which reference is hereby explicitly made.

The nanoemulsions according to the invention and also the preparations according to the invention can also comprise insoluble photoprotective pigments, namely finely disperse metal oxides and salts. Examples of suitable metal oxides are in particular zinc oxide and titanium dioxide and also oxides of iron, zirconium, silicon, manganese, aluminum and cerium, and mixtures thereof. Salts which can be used are silicates (talc), barium sulfate or zinc stearate. The oxides and salts are used in the form of the pigments for skin care and skin-protecting emulsions and also for decorative cosmetics. The particles should have an average diameter of less than 100 nm, preferably between 5 and 50 nm and in particular between 15 and 30 nm. They can have a spherical shape, although it is also possible to use those particles which have an ellipsoidal shape or a shape which deviates in some other way from the spherical configuration. The pigments can also be present in surface-treated, i.e. hydrophilized or hydrophobicized, form. Typical examples thereof are coated titanium dioxides, such as, for example, Titanium dioxide T 805 (Degussa) or Eusolex® T, Eusolex® T-2000, Eusolex® T-Aqua, Eusolex® AVO, Eusolex® T-ECO, Eusolex® T-OLEO and Eusolex® T-S (Merck). Typical examples are zinc oxides, such as, for example, Zinc Oxide neutral, Zinc Oxide NDM (Symrise) or Z-Cote® (BASF) or SUNZnO-AS and SUNZnO-NAS (Sunjun Chemical Co. Ltd.). Suitable hydrophobic coatings here are primarily silicones and specifically trialkoxyoctylsilanes or simethicone. In sunscreen compositions, preference is given to using so-called micropigments or nanopigments. Preference is given to using micronized zinc oxide. Further suitable UV photoprotective filters can be found in the review by P. Finkel in SÖFW Journal 122, 8/1996, pp. 543-548 and Parf. Kosm. 80^{th} volume, No. 3/1999, p. 10 to 16.

Besides the two aforementioned groups of primary photoprotective substances, it is also possible to use secondary photoprotective agents of the antioxidant type, which interrupt the photochemical reaction chain which is triggered when UV radiation penetrates into the skin. Typical examples thereof are amino acids (e.g. glycine, histidine, tyrosine, tryptophan) and derivatives thereof, imidazoles (e.g. urocanic acid) and derivatives thereof, peptides such as D,L-carnosine, D-carnosine, L-carnosine and derivatives thereof (e.g. anserine), carotenoids, carotenes (e.g. -carotene, -carotene, lycopene) and derivatives thereof, chlorogenic acid and derivatives thereof, lipoic acid and derivatives thereof (e.g. dihydrolipoic acid), aurothioglucose, propylthiouracil and other thiols (e.g. thioredoxin, glutathione, cysteine, cystine, cystamine and the glycosyl, N-acetyl, methyl, ethyl, propyl, amyl, butyl and lauryl, palmitoyl, oleyl, linoleyl, cholesteryl and glyceryl esters thereof), and salts thereof, dilauryl thiodipropionate, distearyl thiodipropionate, thiodipropionic acid and derivatives thereof (esters, ethers, peptides, lipids, nucleotides, nucleosides and salts), and sulfoximine compounds (e.g. buthionine sulfoximines, homocysteine sulfoximine, buthionine sulfones, penta-, hexa-, heptathionine sulfoximine) in very low tolerated doses (e.g. pmol to mol/kg), also (metal) chelating agents (e.g. α-hydroxyfatty acids, palmitic acid, phytic acid, lactoferrin), α-hydroxy acids (e.g. citric acid, lactic acid, malic acid), humic acid, bile acid, bile extracts, bilirubin, biliverdin, EDTA, EGTA and derivatives thereof, unsaturated fatty acids and derivatives thereof (e.g. gamma-linolenic acid, linoleic acid, oleic acid), folic acid and derivatives thereof, ubiquinone and ubiquinol and derivatives thereof, vitamin C and derivatives (e.g. ascorbyl palmitate, Mg ascorbyl phosphate, ascorbyl acetate), tocopherols and derivatives (e.g. vitamin E acetate), vitamin A and derivatives (vitamin A palmitate), and coniferyl benzoate of benzoin resin, rutinic acid and derivatives thereof, α-glycosylrutin, ferulic acid, furfurylideneglucitol, carnosine, butylhydroxytoluene, butylhydroxyanisole, nordihydroguaicic acid, nordihydroguaiaretic acid, trihydroxybutyrophenone, uric acid and derivatives thereof, mannose and derivatives thereof, superoxide dismutase, zinc and derivatives thereof (e.g. ZnO, ZnSO4), selenium and derivatives thereof (e.g. selenomethionine), stilbenes and derivatives thereof (e.g. stilbene oxide, trans-stilbene oxide) and the derivatives (salts, esters, ethers, sugars, nucleotides, nucleosides, peptides and lipids) suitable according to the invention of these specified active ingredients.

In one preferred embodiment of the invention, the nanoemulsions or the preparations according to the invention comprise at least one UV photoprotective filter selected from the group consisting of 4-methyl-benzylidenecamphor, benzophenone-3, butylmethoxy-dibenzoylmethane, bis-ethylhexyloxyphenol methoxyphenyl triazine, methylene bis-benzotriazolyl tetramethylbutylphenol, diethylhexyl butamido triazone, ethylhexyl triazone and diethylamino hydroxybenzoyl hexyl benzoate, 3-(4′-trimethylammonium)benzylidene-bornan-2-one methyl sulfate, 3,3′-(1,4-phenylene-dimethine)bis(7,7-dimethyl-2-oxobicyclo[2.2.1]heptane-1-methanesulfonic acid) and its salts, 3-(4′-sulfo)-benzylidenebornan-2-one and its salts, polymer of N-{(2 and 4)-[2-oxoborn-3-ylidene)methyl}benzyl]acrylamide, 2-(2H-benzotriazol-2-yl)-4-methyl-6-(2-methyl-3-(1,3,3,3-tetramethyl-1-(trimethylsilyloxy)disiloxanyl)-propyl)phenol, dimethicodiethyl benzalmalonates and their mixtures.

These UV photoprotective filters are commercially available, for example, under the following trade names:

NeoHeliopan®MBC (INCI: 4-Methylbenzylidene Camphor; manufacturer: Symrise); NeoHeliopan®BB (INCI: Benzophenone-3, manufacturer: Symrise); Parsol®1789 (INCI: Butyl Methoxydibenzoylmethane, manufacturer: Hoffmann-La Roche (Givaudan); Tinosorb®S (INCI: Bis-Ethylhexyloxyphenol Methoxyphenyl Triazine); Tinosorb®M (INCI: Methylene Bis-Benzotriazolyl Tetramethylbutyl-phenol); manufacturer: Ciba Specialty Chemicals Corporation; Uvasorb®HEB (INCI: Diethylhexyl Butamido Triazone, manufacturer: 3V Inc.), Uvinul® 150 (INCI: Ethylhexyl Triazone, manufacturer: BASF AG); Uvinul® A plus (INCI: Diethylamino Hydroxybenzoyl Hexyl Benzoate: manufacturer: BASF AG; Mexoryl® SO: 3-(4′-trimethylammonium)benzylidenebornan-2-one methyl sulfate, INCI: Camphor Benzalkonium Methosulfate; Mexoryl®SX: 3,3′-(1,4-phenylenedimethine)bis(7,7-dimethyl-2-oxobicyclo[2.2.1]heptane-1-methanesulfonic acid), CTFA: INCI Terephthalylidene Dicamphor Sulfonic Acid; Mexoryl® SL: 3-(4′-sulfo)benzylidenebornan-2-one, INCI Benzylidene Camphor Sulfonic Acid; Mexoryl®SW: polymer of N-{(2 and 4)-[2-oxoborn-3-ylidene)methyl}-benzyl]acrylamide, INCI Polyacrylamidomethyl Benzylidene Camphor; Mexory®SL: 2-(2H-benzotriazol-2-yl)-4-methyl-6-(2-methyl-3-(1,3,3,3-tetramethyl-1-(trimethylsilyloxy)disiloxanyl)propyl)phenol; INCI: DROMETRIZOLE TRISILOXANE; Parsol® SLX: dimethicodiethyl benzalmalonate, INCI Polysilicone-15.

The nanoemulsions according to the invention and the preparations according to the invention can comprise the UV photoprotective filters in amounts of from 0.5 to 30% by weight, preferably 2.5 to 20% by weight, particularly preferably 5-15% by weight—based on the nanoemulsion or based on the preparation according to the invention.

Surprisingly, it has been found that the nanoemulsions according to the invention are particularly suitable for solubilizing oil-soluble vitamins.

In one preferred embodiment of the invention, the nanoemulsions comprise at least one vitamin, preferably an oil-soluble vitamin.

Surprisingly, it has been found that the ether mixtures of the formula (I-C) according to the invention are particularly suitable for solubilizing vitamins, preferably oil-soluble vitamins.

The invention provides preparations comprising alkyl and/or alkenyl ether mixture of alkyl and/or alkenyl polyglycosides of the formula (I-C)

(Gₘ-R¹)R²ₙ (I-C)
in which G is a sugar radical having 5 or 6 carbon atoms,
R¹ is a C6 to C22 alkyl and/or alkenyl radical in acetal bond,
R² is a C1 to C4 alkyl and/or alkenyl group in ether bond,
m is an average value from 1.2 to 1.8, and
n is a number from 1.4 to 2.6,
   where at least 50% by weight of the alkyl and/or alkenyl ethers comprise a radical R¹ with a carbon chain greater than or equal to 12, and at least one vitamin, preferably an oil-soluble vitamin.

Suitable oil-soluble vitamins which may be mentioned are vitamin A, vitamin D, vitamin E and vitamin K. Furthermore, L-ascorbyl palmitate may be mentioned. A particularly preferred oil-soluble vitamin is vitamin E. The term vitamin E is a collective name for α-tocopherol, β-tocopherol, γ-tocopherol, δ-tocopherol and α-tocotrienol, β-tocotrienol, γ-tocotrienol and δ-tocotrienol. Also included are the respective tocopherol acetates and tocopherol palmitates.

Suitable water-soluble vitamins which may be mentioned are L-ascorbic acid (vitamin C), and the vitamins of the B group: thiamine (vitamin B₁), riboflavin (vitamin B₂, vitamin G), niacin (vitamin B₃), pantothenic acid (vitamin B₃, vitamin B₅), vitamin B₆, biotin (vitamin B₇, vitamin H), folic acid (vitamin B₉, vitamin B_{c} or vitamin M) and vitamin B₁₂.

Surprisingly, it has been found that the nanoemulsions according to the invention are particularly suitable for solubilizing perfume oils.

In one preferred embodiment of the invention, the nanoemulsions comprise at least one perfume oil, preferably an oil-soluble perfume oil.

Surprisingly, it has been found that the ether mixtures of the formula (I-C) according to the invention are particularly suitable for solubilizing perfume oils, in particular oil-soluble perfume oils.

The invention provides preparations comprising alkyl and/or alkenyl ether mixture of alkyl and/or alkenyl polyglycosides of the formula (I-C)

(Gₘ-R¹)R²ₙ (I-C)
in which G is a sugar radical having 5 or 6 carbon atoms,
R¹ is a C6 to C22 alkyl and/or alkenyl radical in acetal bond,
R² is a C1 to C4 alkyl and/or alkenyl group in ether bond,
m is an average value from 1.2 to 1.8, and
n is a number from 1.4 to 2.6,
   where at least 50% by weight of the alkyl and/or alkenyl ethers comprise a radical R¹ with a carbon chain greater than or equal to 12, and at least one perfume oil, preferably an oil-soluble perfume oil.

The term “perfume oils” encompasses individual natural or synthetic fragrances and also mixtures of natural or synthetic fragrances, aromas and mixtures of fragrances and aromas. Natural fragrances are extracts from flowers (lily, lavender, rose, jasmine, neroli, ylang ylang), stems and leaves (geranium, patchouli, petitgrain), fruits (anise, coriander, caraway, juniper), fruit peels (bergamot, lemon, orange), roots (mace, angelica, celery, cardamom, costus, iris, calmus), woods (pine wood, sandalwood, guaiac wood, cedar wood, rosewood), herbs and grasses (tarragon, lemongrass, sage, thyme), needles and branches (spruce, fir, pine, dwarf-pine), resins and balsams (galbanum, elemi, benzoin, myrrh, olibanum, opoponax). Also suitable are animal raw materials, such as, for example, civet and castoreum. Typical synthetic fragrance compounds are products of the ester, ether, aldehyde, ketone, alcohol and hydrocarbon types. Fragrance compounds of the ester type are, for example, benzyl acetate, phenoxyethyl isobutyrate, p-tert-butyl cyclohexylacetate, linalyl acetate, dimethylbenzyl-carbinyl acetate, phenylethyl acetate, linalyl benzoate, benzyl formate, ethylmethyl phenylglycinate, allyl cyclohexylpropionate, styrallyl propionate and benzyl salicylate. The ethers include, for example, benzyl ethyl ethers, the aldehydes include, for example, the linear alkanals having 8 to 18 carbon atoms, citral, citronellal, citronellyloxyacetaldehyde, cyclamenaldehyde, hydroxycitronellal, lilial and bourgeonal, the ketones include, for example, the ionones, α-isomethylionone and methyl cedryl ketone, the alcohols include anethol, citronellol, eugenol, isoeugenol, geraniol, linalool, phenylethyl alcohol and terpineol, the hydrocarbons include primarily the terpenes and balsams. However, preference is given to using mixtures of different fragrances which together produce a pleasing scent note. Essential oils of relatively low volatility, which in most cases are used as aroma components, are also suitable as perfume oils, e.g. sage oil, chamomile oil, oil of cloves, melissa oil, mint oil, cinnamon leaf oil, linden blossom oil, juniper berry oil, vetiver oil, olibanum oil, galbanum oil, labolanum oil and lavandin oil. Bergamot oil, dihydromyrcenol, lilial, lyral, citronellol, phenyl-ethyl alcohol, α-hexylcinnamaldehyde, geraniol, benzyl acetone, cyclamenaldehyde, linalool, boisambrene forte, ambroxan, indole, hedione, sandelice, lemon oil, mandarin oil, orange oil, allyl amyl glycolate, cyclovertal, lavandin oil, clary sage oil, β-damascone, geranium oil bourbon, cyclohexyl salicylate, Vertofix Coeur, Iso-E-Super, Fixolide NP, evernyl, iraldein gamma, phenylacetic acid, geranyl acetate, benzyl acetate, rose oxide, romillat, irotyl and floramat, alone or in mixtures, are preferably used.

Suitable aromas are, for example, peppermint oil, spearmint oil, anise oil, star anise oil, caraway oil, eucalyptus oil, fennel oil, lemon oil, wintergreen oil, clove oil, menthol and the like.

Depending on the intended application, the nanoemulsions and the preparations according to the invention comprise a series of auxiliaries and additives, such as, for example, pearlescent waxes, consistency regulators, thickeners, superfatting agents, stabilizers, polymers, fats, waxes, lecithins, phospholipids, biogenic active ingredients, antidandruff agents, film formers, swelling agents, insect repellents, self-tanning agents, tyrosinase inhibitors (depigmentation agents), hydrotropes, further solubilizers, preservatives, dyes etc.

The invention provides preparations comprising alkyl and/or alkenyl ether mixture of alkyl and/or alkenyl polyglycosides of the formula (I-C)

(Gₘ-R¹)R²ₙ (I-C)
in which G is a sugar radical having 5 or 6 carbon atoms,
R¹ is a C6 to C22 alkyl and/or alkenyl radical in acetal bond,
R² is a C1 to C4 alkyl and/or alkenyl group in ether bond,
m is an average value from 1.2 to 1.8, and
n is a number from 1.4 to 2.6,
   where at least 50% by weight of the alkyl and/or alkenyl ethers comprise a radical R¹ with a carbon chain greater than or equal to 12, and at least one compound selected from the group consisting of polymers, preservatives, antiperspirant/deodorant active ingredients, self-tanning agents, dyes, stabilizers, hydrotropes, biogenic active ingredients, insect repellent and/or tyrosinase inhibitors.

In one embodiment of the invention, the nanoemulsions according to the invention and the preparations according to the invention comprise at least one polymer. Suitable polymers are, for example, cationic, anionic, zwitterionic, amphoteric and nonionic polymers.

Suitable cationic polymers are, for example, cationic cellulose derivatives, such as, for example, a quaternized hydroxyethylcellulose, which is available under the name Polymer JR 400® from Amerchol, cationic starch, copolymers of diallylammonium salts and acrylamides, quaternized vinylpyrrolidone/vinyl-imidazole polymers, such as, for example, Luviquat® (BASF), condensation products of polyglycols and amines, quaternized collagen polypeptides, such as, for example, lauryldimonium hydroxypropyl hydrolyzed collagen (Lamequat® L/Grünau), quaternized wheat polypeptides, polyethylenimine, cationic silicone polymers, such as, for example, amidomethicones, copolymers of adipic acid and dimethylaminohydroxy-propyldiethylenetriamine (Cartaretine®/Sandoz), copolymers of acrylic acid with dimethyldiallylammonium chloride (Merquat® 550/Chemviron), polyaminopolyamides, cationic chitin derivatives such as, for example, quaternized chitosan, optionally in microcrystalline distribution, condensation products of dihaloalkylene, such as, for example, dibromobutane with bisdialkylamines, such as, for example, bisdimethyl-amino-1,3-propane, cationic guar gum, such as, for example, Jaguar® CBS, Jaguar® C-17, Jaguar® C-16 from Celanese, quaternized ammonium salt polymers, such as, for example, Mirapol® A-15, Mirapol® AD-1, Mirapol® AZ-1 from Miranol.

Suitable anionic, zwitterionic, amphoteric and nonionic polymers are, for example, vinyl acetate/crotonic acid copolymers, vinylpyrrolidone/vinyl acrylate copolymers, vinyl acetate/butyl maleate/isobornyl acrylate copolymers, methyl vinyl ether/maleic anhydride copolymers and esters thereof, uncrosslinked polyacrylic acids and polyacrylic acids crosslinked with polyols, acrylamidopropyltrimethylammonium chloride/acrylate copolymers, octylacrylamide/methyl methacrylate/tert-butylaminoethyl methacrylate/2-hydroxypropyl methacrylate copolymers, polyvinylpyrrol-idone, vinylpyrrolidone/vinyl acetate copolymers, vinylpyrrolidone/dimethylaminoethyl methacrylate/vinyl-caprolactam terpolymers and optionally derivatized cellulose ethers and silicones.

Likewise suitable polymers are polysaccharides, in particular xanthan gum, guar guar, agar agar, alginates and tyloses and also, for example, Aerosil grades (hydrophilic silicas), carboxymethylcellulose and hydroxyethylcellulose and hydroxypropylcellulose, poly-vinyl alcohol, polyvinylpyrrolidone and bentonites, such as, for example, Bentone® Gel VS-5PC (Rheox).

Likewise suitable are so-called quaternary polymers, e.g. with the INCI name Polyquaternium-37, which conform to the following general formula:

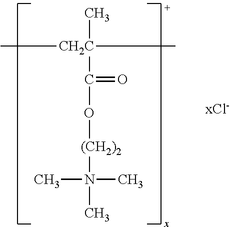

Alternatively, it is also possible to use other dialkylaminoalkyl (meth)acrylates and their ammonium salts or dialkylaminoalkyl(meth)acrylamides obtainable by alkylation or protonation, and also their ammonium salts obtainable by alkylation or protonation. Particular preference is given to polymers comprising MAPTAC, APTAC, MADAME, ADAME, DMAEMA and TMAEMAC. Moreover, it is also possible to use copolymers with anionic, further cationic or uncharged monomers in accordance with the invention, in particular those which, besides the specified alkylaminoalkyl (meth)acrylate or -(meth)acrylamide monomers, additionally comprise (meth)acrylic acid and/or 2-acrylamido-2-methylpropanesulfonic acid and/or acrylamide and/or vinylpyrrolidone and/or alkyl (meth)-acrylates.

By way of example, mention may be made of those polymers with the INCI name Polyquaternium-11, Polyquaternium-13, Polyquaternium-14, Polyquaternium-15, Polyquaternium-28, Polyquaternium-32, Polyquaternium-43, Polyquaternium-47.

In one embodiment of the invention, the nanoemulsions according to the invention and the preparations according to the invention comprise at least one preservative. Suitable preservatives are, for example, phenoxyethanol, formaldehyde solution, parabens, pentanediol or sorbic acid, and also the silver complexes known under the name Surfacine®. Furthermore, suitable preservatives are the 1,2-alkanediols having 5 to 8 carbon atoms described in WO 07/048,757.

Suitable preservatives are in particular the substances approved according to Annex VI of the Commission Directive (in the version Commission Directive 2007/22/EC of 17 Apr. 2007 amending Council Directive 76/768/EEC, concerning cosmetic products, for the purposes of adapting Annexes IV and VI thereto to technical progress), to which reference is hereby explicitly made.

In one embodiment of the invention, the nanoemulsions according to the invention or the preparations according to the invention comprise at least one antiperspirant/deodorant active ingredient.

According to the invention, suitable antiperspirant-/deodorant active ingredients are all active ingredients which counteract, conceal or eliminate body odors. Body odors are formed as a result of the action of skin bacteria on apocrine perspiration, with the formation of unpleasant smelling degradation products. Suitable antiperspirant/deodorant active ingredients are in particular compounds selected from the group consisting of antiperspirants, esterase inhibitors, bactericidic or bacteriostatic active ingredients and/or perspiration-absorbing substances.

### Antiperspirants

Antiperspirants are salts of aluminum, zirconium or zinc. Such suitable antihydrotic active ingredients are, for example, aluminum chloride, aluminum chlorohydrate, aluminum dichlorohydrate, aluminum sesquichlorohydrate and complex compounds thereof e.g. with 1,2-propylene glycol. Aluminum hydroxy-allantoinate, aluminum chloride tartrate, aluminum zirconium trichlorohydrate, aluminum zirconium tetrachlorohydrate, aluminum zirconium pentachloro-hydrate and complex compounds thereof e.g. with amino acids such as glycine. Preference is given to using aluminum chlorohydrate, aluminum zirconium tetrachloro-hydrate, aluminum zirconium pentachlorohydrate and complex compounds thereof.

The nanoemulsions according to the invention or the preparations according to the invention can comprise the antiperspirants in amounts of from 1 to 50, preferably 5 to 30 and in particular 8 to 25% by weight—based on the nanoemulsion or based on the preparation according to the invention.

### Esterase Inhibitors

In the presence of perspiration in the axillary area, extracellular enzymes—esterases, preferably proteases and/or lipases—are formed by bacteria; these cleave esters present in the perspiration and thereby release odorous substances. Suitable esterase inhibitors are preferably trialkyl citrate such as trimethyl citrate, tripropyl citrate, triisopropyl citrate, tributyl citrate and in particular triethyl citrate (Hydagen®CAT, Cognis GmbH, Düsseldorf/FRG). The substances inhibit the enzyme activity and thereby reduce the formation of odor. Further substances which are suitable as esterase inhibitors are sterol sulfates or phosphates, such as, for example, lanosterol, cholesterol, campesterol, stigmasterol and sitosterol sulfate and phosphate, dicarboxylic acids and esters thereof, such as, for example, glutaric acid, monoethyl glutarate, diethyl glutarate, adipic acid, monoethyl adipate, diethyl adipate, malonic acid and diethyl malonate, hydroxycarboxylic acids and esters thereof, such as, for example, citric acid, malic acid, tartaric acid or diethyl tartrate, and zinc glycinate.

The nanoemulsions according to the invention or the preparations according to the invention can comprise the esterase inhibitors in amounts of from 0.01 to 20, preferably 0.1 to 10 and in particular 0.3 to 5% by weight—based on the nanoemulsion or based on the preparation according to the invention.

### Bactericidic and Bacteriostatic Active Ingredients

Typical examples of suitable bactericidic and bacteriostatic active ingredients are in particular chitosan and phenoxyethanol. 5-Chloro-2-(2,4-dichloro-phenoxy)phenol has also proven particularly effective; this is sold under the name Irgasan® by Ciba-Geigy, Basel/CH. Suitable germicidal agents are in principle all substances that are effective against Gram-positive bacteria, such as, for example, 4-hydroxybenzoic acid and its salts and esters, N-(4-chlorophenyl)-N′-(3,4-dichlorophenyl)urea, 2,4,4′-trichloro-2′-hydroxy-diphenyl ether (triclosan), 4-chloro-3,5-dimethylphenol, 2,2′-methylenebis(6-bromo-4-chloro-phenol), 3-methyl-4-(1-methylethyl)phenol, 2-benzyl-4-chlorophenol, 3-(4-chlorophenoxy)-1,2-propanediol, 3-iodo-2-propynyl butylcarbamate, chlorhexidine, 3,4,4′-trichlorocarbanilide (TTC), antibacterial fragrances, thymol, thyme oil, eugenol, clove oil, menthol, mint oil, farnesol, phenoxyethanol, glycerol monocaprate, glycerol monocaprylate, glycerol monolaurate (GML), diglycerol monocaprate (DMC), N-alkylamides of salicylic acid, such as, for example, N-n-octylsalicyl-amide or N-n-decylsalicylamide. The nanoemulsions according to the invention or the preparations according to the invention can comprise the bactericidic or bacteriostatic active ingredients in amounts of from 0.01 to 5 and preferably 0.1 to 2% by weight—based on the nanoemulsions or based on the preparation according to the invention.

### Perspiration-Absorbing Substances

Suitable perspiration-absorbing substances are modified starch, such as, for example, Dry Flo Plus (National Starch), silicates, talc and other substances of similar modification which appear to be suitable for the absorption of perspiration.

The nanoemulsions according to the invention and the preparations according to the invention can comprise the perspiration-absorbing substances in amounts of from 0.1 to 30, preferably 1 to 20 and in particular 2 to 8% by weight—based on the nanoemulsions or based on the preparation according to the invention.

In one embodiment of the invention, the nanoemulsions according to the invention or the preparations according to the invention comprise at least one self-tanning agent.

Self-tanning agents are to be understood as meaning substances which cause tanning of the skin. By way of example, mention may be made of alpha,beta-unsaturated aldehydes which react with the amino acids in the skin in the sense of a Maillard reaction to give colored compounds. Further suitable active ingredients for self-tanning agents are natural or synthetic ketols and aldols. Suitable active ingredients which may be mentioned by way of example are dihydroxyacetone, erythrulose glycerol aldehyde, alloxan, hydroxymethyl-glyoxal, gamma-dialdehyde, 6-aldo-D-fructose, ninhydrin and meso-tartardialdehyde. Suitable self-tanning agents are in particular dihydroxyacetone and/or erythrulose.

Mixtures of the aforementioned active ingredients with one another or with mucondialdehyde and/or naphthoquinones such as, for example, 5-hydroxy-1,4-naphthoquinone (juglone) and 2-hydroxy-1,4-naphthoquinone have proven to be particularly advantageous. The nanoemulsions according to the invention and the preparations according to the invention comprise the self-tanning agents usually in concentrations of from 1 to 10, in particular from 2 to 5% by weight—based on the nanoemulsion or based on the preparation according to the invention.

In one embodiment of the invention, the nanoemulsions and the preparations according to the invention comprise at least one dye.

The dyes may be either of synthetic origin or of natural origin. A list of suitable dyes can be found in EP 1 371 359 A2, p. 8, 11. 25-57, p. 9 and p. 10 and also p. 11, 11. 1 to 54, to which reference is hereby explicitly made. The nanoemulsions according to the invention and the preparations according to the invention comprise usually 0.01 to 5% by weight, preferably 0.1 to 1.0% by weight, of dyes—based on the nanoemulsion or based on the preparation according to the invention.

Suitable dyes are in particular the dyes approved according to Annex IV of the Commission Directive (in the version: Commission Directive 2007/22/EC of 17 Apr. 2007 amending Council Directive 76/768/EEC, concerning cosmetic products, for the purposes of adapting Annexes IV and VI thereto to technical progress), to which reference is hereby explicitly made.

In one embodiment of the invention, the nanoemulsions according to the invention and the preparations according to the invention comprise at least one stabilizer. Stabilizers which can be used are metal salts of fatty acids, such as, for example, magnesium, aluminum and/or zinc stearates and ricinoleates.

In one embodiment of the invention, the nanoemulsions according to the invention and the preparations according to the invention comprise at least one hydrotrope. To improve the flow behavior, hydrotropes, such as, for example, ethanol, isopropyl alcohol, or polyols, can also be used. Polyols which are suitable here preferably have 2 to 15 carbon atoms and at least two hydroxyl groups. The polyols can also contain further functional groups, in particular amino groups, and/or be modified with nitrogen.

In one embodiment of the invention, the nanoemulsions according to the invention and the preparations according to the invention comprise at least one biogenic active ingredient. Biogenic active ingredients are to be understood as meaning, for example, (deoxy)ribonucleic acid and fragmentation products thereof, β-glucans, bisabolol, allantoin, phytantriol, panthenol, AHA acids, amino acids, ceramides, pseudoceramides, essential oils, plant extracts, such as, for example, aloe vera, prune extract, bambara nut extract.

In one embodiment of the invention, the nanoemulsions according to the invention and the preparations according to the invention comprise at least one insect repellent. Suitable insect repellents are, for example, N,N-diethyl-m-toluamide, 1,2-pentanediol or ethyl 3-(N-n-butyl-N-acetylamino)propionate, which is sold under the name Insect Repellent® 3535 by Merck KGaA, and also butylacetylaminopropionates.

In one embodiment of the invention, the nanoemulsions according to the invention and the preparations according to the invention comprise at least one tyrosinase inhibitor. Suitable tyrosinase inhibitors, which prevent the formation of melanin and are used in depigmentation agents, are, for example, arbutin, ferulic acid, kojic acid, coumaric acid and ascorbic acid (vitamin C).

### EXAMPLES

### Preparation Example 1

290 g (0.36 mol) of Plantacare® 1200, 227 g (2.1 mol) of NaOH (37% strength) and 30 g of isopropanol were initially introduced in a suitable pressurized reactor and heated to 85° C. Then, at a pressure of at most 3.3 bar, 70 g (1.39 mol) of methyl chloride were metered in in portions. After a metering time of 9 hours, the reaction was complete. To remove the NaCl formed during the reaction, the product was freeze-dried and extracted several times with ethanol. The combined extracts were freed from the ethanol on a rotary evaporator and taken up in 70% water.

The resulting clear product had a degree of polymerization m of 1.4; a fraction of 90% by weight alkyl ethers with a carbon chain greater than or equal to 12 based on the alkyl ether mixture, and a degree of methylation n=1.7.

### Preparation Example 2

290 g (0.36 mol) of Plantacare® 1200 and 302 g (2.8 mol) of NaOH (37% strength) were initially introduced in a suitable pressurized reactor and heated to 85° C. Then, at a pressure of at most 3.3 bar, 110 g (2.18 mol) of methyl chloride were metered in in portions. After a metering time of 12 hours, the reaction was complete. To remove the NaCl formed during the reaction, the product was freeze-dried and extracted several times with ethanol. The combined extracts were freed from the ethanol on a rotary evaporator and taken up in 70% water. The resulting clear product had a degree of methylation of 2.5.

The resulting clear product had a degree of polymerization m of 1.4; a fraction of 90% by weight alkyl ethers with a carbon chain greater than or equal to 12, based on the alkyl ether mixture, and also a degree of methylation n=2.5.

### Preparation Example 3

1700 g (2.08 mol) of Plantacare® 1200, 1700 g of water and 832.2 g (10.4 mol) of NaOH (50% strength) were initially introduced in a suitable pressurized reactor and heated to 60° C. Then, at a pressure of at most 5 bar, 525.3 g (10.4 mol) of methyl chloride were metered in in portions. After a metering time of 10 hours, the reaction was complete. To remove the NaCl formed during the reaction, the product was freeze-dried and extracted several times with ethanol. The combined extracts were freed from the ethanol on a The resulting clear product had a degree of polymerization m of 1.4; a fraction of 90% by weight alkyl ethers with a carbon chain greater than or equal to 12, based on the alkyl ether mixture, and also a degree of methylation n=1.7.

The Plantacare® 1200 (Cognis) used in the examples is an alkyl polyglucoside (50% by weight active substance) with an average degree of polymerization of 1.2-1.4. The carbon chain distribution in the alkyl polyglucoside is as follows: C8 0-3%, C10 0-4%, C12 67-75%, C14 23-30%, C16 0-2%.

### Comparative Example

247.4 g (0.4 mol) of Glucopon® 215 CSUP (alkyl polyglucosides based on C8 and C10 fatty alcohols, degree of polymerization 1.5), 400 g of water and 320 g (4.0 mol) of NaOH (50% strength) were initially introduced in a suitable pressurized reactor and heated at 80° C. for 2 hours. Then, at 60° C. and a pressure of at most 5 bar, 202.0 g (4.0 mol) of methyl chloride were metered in in portions. After a metering time of 7 hours, the reaction was complete. To remove the NaCl formed during the reaction, the product was freeze-dried and extracted several times with ethanol. The combined extracts were freed from the ethanol on a rotary evaporator and taken up in 70% water.

The resulting clear product had a degree of polymerization m of 1.5; a fraction of less than 5% by weight alkyl ethers with a carbon chain greater than or equal to 12, based on the alkyl ether mixture, and also a degree of methylation of n=2.5.

### Solubilizing Properties

The solubilizing property was investigated as follows: the amount of solubilizer which is required to obtain a clear solution of a 1% strength by weight solution of vitamin E (Copherol® 1250C, Cognis) or of a UV photoprotective filter (Neo Heliopan AV, INCI: Ethylhexyl Methoxycinnamate) in water/ethanol (75/25) was determined. The lesser the amount of solubilizer required to obtain a clear solution, the better the solubilizing properties a substance has. Table 1 shows the results:

Examples 1, 2 and 3 are in accordance with the invention, Example C¹ is the comparative example. In each case, the amounts of solubilizer in % by weight which are required to obtain a clear solution are given.

| | Degree of | 1% by weight | |
|---|---|---|---|
| | methylation | Copherol ® | 1% by weight |
| Example No. | (n) | 1250C | Neo Heliopan |
| Preparation | 1.7 | 4% | 5% |
| Example 1 | | | |
| Preparation | 2.5 | 5% | 5% |
| Example 2 | | | |
| Preparation | 1.7 | 6% | 5% |
| Example 3 | | | |
| Comparative | 2.5 | >7% | >7% |
| Example | | | |

### Preparation of a Nanoemulsion

A mixture of 4.9 g of APG methyl ether as in Preparation Example 3, 0.7 g of cetearyl alcohol, 47.2 g of octane and 47.2 g of demineralized water were poured into a glass jacketed vessel and heated to a temperature of 70-80° C. with stirring (IKA Eurostar Digital, 600 rpm). The heating of the glass jacketed vessel takes place here using a programmable cryostat (Haake P1-C35P). During the preparation process, electrical conductivity and temperature were measured using a conductometer (WTW ProfiLine LF 197-S) and a measurement probe (WTW TertraCon 325/S). This mixture exhibits no conductivity at temperatures above 65° C., is thus in the form of a W/0 emulsion, the phase inversion temperature is exceeded. Cooling to room temperature then takes place, the cooling rate was 0.7° C./min. At the start of the inversion from W/O to O/W, the conductivity starts to increase. After reaching room temperature, a bluish nanoemulsion with an average particle size around 200 nm is present.

What is claimed is:

1-15. (canceled)

16. A nanoemulsion comprising:
(a) an aqueous phase,
(b) an oil phase, and
(c) at least one alkyl and/or alkenyl ether of alkyl and/or alkenyl (poly)glycosides, having formula (I-A):
(Gₘ-R¹)R²ₙ (I-A)
wherein G is a sugar moiety having 5 or 6 carbon atoms,
R¹ is C6-C22 alkyl and/or alkenyl bound via the sugar acetal moiety,
R² is C1-C4 alkyl and/or alkenyl bound as a sugar ether,
m is an average value from 1.0 to 3.0, and
n is a number from 0.5 to 5.0.

17. The nanoemulsion of claim 16, comprising at least one alkyl and/or alkenyl ether mixture of alkyl and/or alkenyl (poly)glycosides, having formula (I-B):
(Gₘ-R¹)R²ₙ (I-B)
wherein G is a sugar moiety having 5 or 6 carbon atoms, R¹ is C6-C22 alkyl and/or alkenyl bound via the sugar acetal moiety,
R² is C1-C4 alkyl and/or alkenyl bound as a sugar ether,
m is an average value from 1.0 to 3.0, and
n is a number from 0.5 to 5.0,
wherein at least 50% by weight of said compounds of formula (I-B) comprise an R¹ group having 12 or more carbon atoms.

18. The nanoemulsion of claim 16, comprising at least one alkyl and/or alkenyl ether mixture of alkyl and/or alkenyl polyglycosides, having formula (I-C):
(Gₘ-R¹)R²ₙ (I-C)
wherein G is a sugar moiety having 5 or 6 carbon atoms,
R¹ is C6-C22 alkyl and/or alkenyl bound via the sugar acetal moiety,
R² is C1-C4 alkyl and/or alkenyl bound as a sugar ether,
m is an average value from 1.2 to 1.8, and
n is a number from 1.4 to 2.6,
wherein at least 50% by weight of said compounds of formula (I-C) comprise an R¹ group having 12 or more carbon atoms.

19. The nanoemulsion of claim 16, further comprising at least one coemulsifier.

20. The nanoemulsion of claim 19, containing less than 10% by weight of ethoxylated emulsifiers.

21. The nanoemulsion of claim 19, containing less than 0.5% by weight of ethoxylated emulsifiers.

22. A method of preparing cosmetic and/or pharmaceutical preparations, comprising the step of adding the nanoemulsion of claim 16 to a cosmetic and/or pharmaceutical base.

23. A method of preparing nanoemulsions, comprising the step of adding to an aqueous phase and an oil phase, one or more alkyl and/or alkenyl ethers of alkyl and/or alkenyl (poly)glycosides, having formula (I-A):
(Gₘ-R¹)R²ₙ (I-A)
wherein G is a sugar moiety having 5 or 6 carbon atoms,
R¹ is C6-C22 alkyl and/or alkenyl bound via the sugar acetal moiety,
R² is C1-C4 alkyl and/or alkenyl bound as a sugar ether,
m is an average value from 1.0 to 3.0, and
n is a number from 0.5 to 5.0,
and/or one or more alkyl and/or alkenyl ether mixtures of alkyl and/or alkenyl (poly)glycosides, having formula (I-B):
(Gₘ-R¹)R²ₙ (I-B)
wherein G is a sugar moiety having 5 or 6 carbon atoms,
R¹ is C6-C22 alkyl and/or alkenyl bound via the sugar acetal moiety,
R² is C1-C4 alkyl and/or alkenyl bound as a sugar ether,
m is an average value from 1.0 to 3.0, and
n is a number from 0.5 to 5.0,
wherein at least 50% by weight of said compounds of formula (I-A) and/or formula (I-B) comprise an R¹ group having 12 or more carbon atoms.

24. The method of claim 23, wherein said nanoemulsions are prepared according to the phase inversion temperature (PIT) method.

25. An alkyl and/or alkenyl ether mixture of alkyl and/or alkenyl polyglycosides, having formula (I-C):
(Gₘ-R¹)R²ₙ (I-C)
wherein G is a sugar moiety having 5 or 6 carbon atoms,
R¹ is C6-C22 alkyl and/or alkenyl bound via the sugar acetal moiety,
R² is C1-C4 alkyl and/or alkenyl bound as a sugar ether,
m is an average value from 1.2 to 1.8, and
n is a number from 1.4 to 2.6,
wherein at least 50% by weight of said compounds of formula (I-C) comprise an R¹ group having 12 or more carbon atoms.

26. A method of preparing alkyl and/or alkenyl ether mixtures of claim 25, comprising the steps of:
(a) providing an alkyl and/or alkenyl polyglycosides of formula (II):
Gₘ-R¹ (II)
wherein G is a sugar moiety having 5 or 6 carbon atoms,
R¹ is C6-C22 alkyl and/or alkenyl bound via the sugar acetal moiety, and
m is an average value from 1.2 to 1.8,
wherein at least 50% by weight of the alkyl and/or alkenyl polyglycosides comprise an R¹ group having 12 or more carbon atoms, and
(b) reacting said compound of formula (II) with an alkylating agent of formula (III):
R²—X (III)
wherein X is a nucleophilic leaving group, and
R² is C1-C4 alkyl and/or alkenyl.

27. A method of solubilizing a lipophilic compound, comprising the step of adding the alkyl and/or alkenyl ether mixture of claim 25 to a mixture comprising said lipophilic compound and an aqueous phase, wherein said lipophilic compound is solubilized in said aqueous phase.

28. A method of preparing a cosmetic and/or pharmaceutical preparation, comprising the step of adding the alkyl and/or alkenyl ether mixture of claim 25 to a cosmetic and/or pharmaceutical base.

29. The method of claim 28, wherein said cosmetic and/or pharmaceutical preparation is in the form of a nanoemulsion.

30. A cosmetic and/or pharmaceutical preparation comprising 0.1% to 20% by weight of alkyl and/or alkenyl ether mixture of claim 25.

31. The cosmetic and/or pharmaceutical preparation of claim 30, further comprising one or more compounds selected from the group consisting of UV photoprotective filters, vitamins, perfume oils and mixtures thereof.

32. A cosmetic and/or pharmaceutical preparation comprising the nanoemulsion of claim 16, and one or more compounds selected from the group consisting of UV photoprotective filters, vitamins, perfume oils and mixtures thereof.

33. A cosmetic and/or pharmaceutical preparation comprising the alkyl and/or alkenyl ether mixture of claim 25, and one or more compounds selected from the group consisting of oil bodies, coemulsifiers and mixtures thereof.
